(19) United States
(12) Patent Application Publication
(10) Pub. No.: US 2003/0118636 A1
(43) Pub. Date: Jun. 26, 2003

(54) Delivery of small hydrophilic molecules packaged into lipid vesicles

(76) Inventors: Robert H.E. Friesen, Haren (NL); Berend Poolman, Haren (NL); Bernard L. Feringa, Paterswolde (NL); Jan B.F.N. Engberts, Groningen (NL)

Correspondence Address: TRASK BRITT, P.O. BOX 2550, SALT LAKE CITY, UT, 84110, US

(21) Appl. No.: 10/281,048

(22) Filed: Oct. 24, 2002

Related U.S. Application Data
(63) Continuation-in-part of application No. PCT/NL02/00412, filed on Jun. 21, 2002.

(30) Foreign Application Priority Data
Jun. 21, 2001 (EP) 01202401.4

(51) Int. Cl. A61K 9/127; C12N 5/00

(52) U.S. Cl. 424/450; 435/325

(57) ABSTRACT

Methods and compositions for the generation of vehicles for delivering small molecules are disclosed. In one aspect, lipid vesicles having a proteinaceous channel and small molecules are generated. The proteinaceous channel and/or the lipid vesicle are formulated such that the small molecule is released in the vicinity of or near a target cell. The target cell may be located in vitro or in vivo.

### CROSS-REFERENCE TO RELATED APPLICATIONS

This application is a continuation in part of PCT/NL02/00412, filed Jun. 21, 2002, the contents of which are incorporated herein in its entirety.

### TECHNICAL FIELD

The present invention relates to the field of medicine. More particularly, the invention relates to the field of pharmaceutics.

### BACKGROUND

Liposomes are typically spherical lipid bilayers ranging in size from about 50 nm to 1000 m in diameter and may serve as convenient delivery vehicles for biologically active molecules. Lipid/drug aggregates are easy to form and are vulnerable to structural manipulations, allowing for the adjustment of their properties for particular purposes. In selected cases, the application of liposomes in pharmacological therapy improves drug pharmacokinetics compared to its free form. The major advantages of the liposome application are: the protection of active compounds from degradation; the increase in circulation time and the possibility to achieve partial or total tissue or cell selectivity. Selectivity improves drug potency, eliminates side effects and allows for dosage reduction.

Although liposome mediated delivery of biologically active molecules is a promising approach, there are also limitations associated with the current forms. A particular problem is the hydrophobic nature of the lipid bilayer of the liposome since hydrophilic drugs are not easily released from liposomes. To effectively release hydrophilic compounds, the integrity of the lipid bilayer must often be disrupted which is often not possible in a controlled fashion. biologically active molecule to an exterior of the lipid vesicle, thus releasing the biologically active molecule from the lipid vesicle.

In one aspect, the invention provides a method for producing a lipid vesicle for modulating the bio-availability of a small hydrophilic molecule upon administration of the small hydrophilic molecule in the lipid vesicle to a subject. For ease of explanation, the terms “small hydrophilic molecule” and “small molecule” may be used interchangeably. The method comprises producing a lipid vesicle comprising the small hydrophilic molecule and a proteinaceous channel, wherein the open state of the proteinaceous channel allows passage of the small hydrophilic molecule to the exterior of the lipid vesicle. As used herein, the terms “lipid vesicle” and “vesicle” may be used interchangeably. For use in a biological system, the produced lipid vesicle may be tested for its biocompatibility and/or for the capability of the small hydrophilic molecule to pass through the proteinaceous channel. Often biocompatibility of the lipid vesicle and capability of the small hydrophilic molecule to pass through the proteinaceous channel can be predicted from the properties of the various components.

The invention also includes the administration of the lipid vesicle to a biological system. In this embodiment, the small hydrophilic molecule comprises a molecule that is biologically active in the biological system. Lipid vesicles produced according to the invention may be used to modulate bio-availability whether the proteinaceous channel is in the open state or in the closed state after production of the lipid vesicles. The open state may be used to load the lipid vesicle with the small hydrophilic molecule. For producing the lipid vesicle, the proteinaceous channel is used in the closed state or is closed upon loading of the lipid vesicle with the small hydrophilic molecule.

The invention further provides a method for making a small hydrophilic molecule bio-available comprising providing a biological system with a lipid vesicle and/or a composition according to the present invention. The method provides an altered release profile for the small hydrophilic molecule compared to the absence of the proteinaceous channel. Bio-availability can further be controlled, or modulated, by providing a signal for activating the proteinaceous channel in the biological system. The signal can be provided to the biological system or to a part thereof. In an illustrated embodiment, the signal is provided to a part of the biological system. In this manner, it is possible to further restrict, or at least in part, limit bio-availability of the small hydrophilic molecule to one or more parts of the biological system as a whole. Signals for activating, or opening, the proteinaceous channel will be discussed herein.

In another embodiment, the invention provides a method for obtaining controlled release of hydrophilic drugs from liposomes, or lipid vesicles. To this end, the present invention provides a method for delivering a small hydrophilic molecule to a cell, comprising loading a lipid vesicle with the small hydrophilic molecule and administering the lipid vesicle to fluid that is in contact with the cell. The lipid vesicle further comprises a proteinaceous channel, wherein the channel in the open state allows passage of the small molecule to the exterior of the vesicle in the vicinity of the cell. The vesicles may be administered to culture medium of cells growing in vitro. The vesicles may also be administered to a subject, such as an animal or a mammal. More preferably, the vesicle is administered to a human. The proteinaceous channel may be any proteinaceous channel that allows passage of the small molecule. The proteinaceous channel may comprise a solute channel which is capable of allowing passage of ions and small hydrophilic molecules. The proteinaceous channel may also comprise an ion channel or a mechanosensitive channel, such as a mechanosensitive channel of large conductance (MscL) or a functional equivalent thereof.

The invention further provides a method for delivering a small hydrophilic molecule to a cell, wherein the method comprises loading a lipid vesicle with the small molecule and administering the lipid vesicle to a subject comprising the cell. The lipid vesicle may further comprise an MscL or a functional equivalent thereof, where the channel in the open state allows passage of the small molecule to the exterior of the vesicle.

In nature, MscL allows prokaryotes, such as bacteria, to rapidly adapt to a sudden change in environmental conditions such as osmolarity. The MscL channel opens in response to increases in membrane tension, which allows for the efflux of cytoplasmic constituents. By allowing passage of the constituents to the outside of the prokaryote, the prokaryote is able to reduce the damage that the sudden change in environmental conditions would have otherwise inflicted. The genes encoding MscL homologues from various prokaryotes are cloned (Moe et al.). Nucleic acid and amino acid sequences are available and have been used to obtain heterologous over-expression of several MscL (Moe et al.). In the present invention, lipid vesicles, such as liposomes, comprising MscL or a functional equivalent thereof are loaded with small hydrophilic molecules whereupon the loaded small hydrophilic molecules may be released from the vesicles upon activation or opening of the channel. Loading of the lipid vesicle may be accomplished in many ways as long as the small molecules are dissolved in a hydrophilic solvent which is separated from the surrounding hydrophilic solvent by a lipid bilayer.

In another embodiment, the lipid vesicle may be formulated to allow preferential opening of the channel near cells of a selected tissue. Since activation of MscL has been found to be controllable, it is possible to tune the type and relative amount of lipids in the vesicle such that the amount of membrane tension required to activate the channel is altered. Thus, depending on the circumstances existing in the vicinity near the cells of the selected tissue, the lipid vesicle may be tuned to allow preferential activation of the channel and thus preferential release of the small hydrophilic molecule in the vicinity of the cells of the selected tissue.

Release of the small hydrophilic molecule near the cell or in the vicinity of the cells is obtained when the small molecules are released in fluid that contacts the cell. For instance, release into culture medium including cells is intended to mean release near or in the vicinity of the cells. The terms “near” or “vicinity” as used herein will be used to refer to a functional distance rather than a physical distance. For instance, release of the small molecule from a lipid vesicle of the invention in a capillary vessel that feeds target cells should be considered as released “near” or in the “vicinity” of the target cells. In contrast, release of the small molecule in blood vessels that carry blood away from the target cells can be as close physically as the release of the small molecules in the capillary vessels that feed the target cells, but is not considered to be released near or in the vicinity of the target cells as the released small molecule is carried away from the target cells. An exception is made for lymph and other similar fluids; although these types of fluids are carried away from target cells, the contact with the surrounding cells is so intense that the small molecule can still exert its effect upon release. If a physical distance is used to define the terms “near” or “in the vicinity of,” the physical distance is not more than 100 times and preferably not more than 20 times the radius of a target cell; more preferably, the distance is not more than 10 times the radius of a target cell.

Compositions comprising lipid vesicles have been used in vivo, for instance, to enable delivery of nucleic acid or anti-tumor drugs to cells. It has been observed that bloodstream administration of such vesicles often leads to the uptake of the lipid vesicles by cells. Uptake of the lipid vesicles by cells appears to correlate with the charge of the lipid in the vesicle. Uptake is particularly a problem with negatively charged lipid vesicles since these vesicles are very quickly removed from the bloodstream by the mononuclear phagocytic system (MPS) in the liver and the spleen. Although the present invention may be used to facilitate uptake of small molecules by cells, it is preferred that the small molecules are delivered to the outside of cells.

In the present invention, it has been found that MscL is also active in lipid vesicles that include positively and/or neutrally charged lipids. Lipid vesicles comprising positively and/or neutrally charged lipids are more resistant to uptake by cells of the MPS. Accordingly, lipid vesicles of the invention may comprise positively and/or neutrally charged lipids. Such lipid vesicles exhibit improved half-lives in the bloodstream and demonstrate improved targeting to non-MPS cells. The lipid part of the lipid vesicles of the present invention directed toward the outside of cells includes positively and/or neutrally charged lipids, thus avoiding cellular uptake through negatively charged lipids and increasing the bloodstream half-life of the lipid vesicles of the present invention. Apart from increasing the half-life of the lipid vesicle in the bloodstream, the positively and/or neutrally charged lipids may also be used to alter the amount of added pressure needed to activate the channel of the lipid vesicle. The lipid vesicles of the present invention, wherein the outwardly directed lipid part of a lipid vesicle includes positively and/or neutrally charged lipids, postpone the rapid cellular uptake as seen with vesicles wherein the outwardly directed part includes negatively charged lipids. Postponed uptake through the MPS system leads to increased circulation times. Apart from this, positively and/or negatively charged lipids may also be used to alter the amount of membrane tension needed to activate the channel.

The signal or event leading to activation of a channel of a lipid vesicle of the present invention may also be changed by altering the MscL in the lipid vesicle. In addition to pH-sensitive MscL mutants, other MscL mutants are available that have a higher probability of being opened when compared to the wild-type MscL derived from Escherichia coli (Bount et al. and Ou et al.). This property can be used to tune the activation potential of the channel in a method or composition of the present invention. For instance, it is known that the pH in tumors is often lower than the pH in the normal tissue surrounding the tumor. Other areas in the body that have a lowered pH are the liver, areas of inflammation and ischemic areas. A lower pH can be used as a trigger to activate the MscL of a lipid vesicle of the present invention. MscL mutants are available that activate, or open, in response to a pH that is frequently encountered in tumors. One non-limiting example of such a pH-sensitive MscL mutant is the G22H mutant. This mutant exhibits a higher open probability at low pH values that are frequently encountered in tumors, as compared to normal pH values of circulating blood (9). Thus, in one embodiment of a method or composition of the present invention, the MscL mutant allows for preferred release of the small molecule in the target tissue.

The small molecule may be any hydrophilic molecule small enough to pass through the pore of a proteinaceous channel of the present invention. The small molecule comprises a diameter of no more than 60 Å, preferably no more than 50 Å and more preferably no more than 40 Å. Particularly, peptides may be used as the small molecules of the present invention. Peptides typically have poor pharmacodynamic properties when injected into the bloodstream. By administering peptides in lipid vesicles of the present invention, it is possible to significantly increase the half-life of peptides in the circulation. Moreover, by enabling controlled release of small molecules using the lipid vesicles of the present invention, it is also possible to have a relatively high bio-availability of the peptide locally, whereas systemically, the bio-availability is low or even absent. This also allows for the therapeutic use of the small molecules locally that are otherwise too toxic when bio-available systemically.

Controlled and/or localized release of small molecules may be achieved in many ways. For instance, the composition of the lipid vesicle and/or the use of a mutant MscL as channels may be varied to control how and where release of the small molecules will occur. In one embodiment, activation of the channel is triggered by a signal. The signal may comprise light, pH, a chemical compound or temperature. Various chemical compounds may be used as long as the chemical compound locally induces opening of the proteinaceous channel. In another embodiment, opening of the channel is induced by providing the chemical compound to a part of the biological system. Non-limiting examples of suitable chemical compounds include compounds that influence the pH of the environment. Another non-limiting example is a compound capable of interacting or reacting with the proteinaceous channel, thus leading to an altered open-probability. A compound that interacts or reacts with the channel may change the gating properties of the channel such that the channel is opened as a result of the compounds interaction or reaction with the channel. In one embodiment, the chemical compound may comprise an MTS molecule, while in another embodiment the chemical compound may comprise reduced glutathione.

As previously discussed herein, the signal for activation of the proteinaceous channel can be exposure of the lipid vesicle to a certain pH, to light or to a certain temperature. Exposure of the channel to the signal can directly or indirectly, such as through an intermediary signal, lead to the activation of the channel. For instance when the signal comprises light, hydrophobic compounds, such as azobenzene phospholipids and related compounds, are available (Song et al.) that mix with the lipids in the liquid vesicle, and upon exposure to light, undergo a structural change that controls the gating of the MscL channel. It is also possible to insert a photosensitive mutant MscL as the channel in the lipid vesicle. Upon exposure to light, a photoreactive molecule conjugated to a specific site of the mutant MscL protein may alter the MscL protein conformation, thus controlling the gating of the MscL channel. Activation through light is just one example of an embodiment wherein opening/activation of the channel is induced by a signal other than membrane tension.

An alteration in the redox-potential is another non-limiting example of a signal that may be used to activate the channel. For instance, MscL can be made sensitive to the local redox-potential after conjugation of a redox-sensitive molecule, such as a nicotinamide adenine dinucleotide derivative, to a specific site of the MscL protein. Such a redox-sensitive MscL may be deactivated by changing the redox-potential of the environment.

Recognition of the open conformation of MscL by an antibody is another non-limiting example where gating of the channel can be induced by a signal other than membrane tension. Such an antibody can be used to preferentially increase the open probability of the channel near target cells. A bispecific antibody comprising the above-mentioned specificity for the open state and specificity for a target cell may be used to accumulate open vesicles near target cells.

Another example of a signal that triggers activation of an MscL is a local anesthetic (Martinac et al.). Local anesthetics work to activate the channel through their incorporation in the lipid bilayer, which changes the properties of the lipid bilayer.

Many other substances exist that may cause activation of the channels. One example is [2-(trimethylammonium)ethyl] methanethiosulfonate bromide (MTSES). This compound is capable of associating with the MscL mutant G22C. Although a hydrophobic moiety at position 22 makes the MscL channel harder to open, a hydrophilic addition at position 22 helps to overcome the mechanical work required to open the MscL channel. The compound MTSES helps to lower the amount of signal required to activate the channel (Yoshimura et al.). Various polar and non-polar variants of MTSES exist that may be used depending on whether the channel should be easier or more difficult to activate.

It is also possible in some applications to change the signal needed for activation of the channel from membrane pressure to another signal. Other signals such as light, local anesthetics, pH, temperature, etc. may be used to facilitate the local delivery of an incorporated small molecule from the lipid vesicle. For instance, through local illumination of an area within the body of a subject with light, a circulating lipid vesicle can be triggered to release incorporated molecules only in the illuminated area of the body. This is a beneficial result of having a signal or an intermediate signal other than pressure for activation of the channel.

In one embodiment, a lipid vesicle of the present invention comprises an asymmetrical bilayer. The asymmetrical bilayer is one example of a method that may be used to tune the lipid vesicle such that the activation of the channel is altered. The force gating the MscL is exerted by the lipid bilayer and amphipaths may generate this force by differential insertion into the two leaflets of the lipid bilayer (Martinac et al.). A signal required for activation of the channel may be provided through an intermediate that is capable of transforming the given signal into a pressure signal, thus allowing the opening of the channel.

Lipid vesicles and/or compositions of the invention including pH-sensitive proteinaceous channels may be used for pH-induced drug release. In tumors and sites of inflammation, the pH of the interstitial fluid is reduced whereas the blood flow is increased and the vasculature is “leaky.” pH-sensitive liposomes have been developed for these purposes (Shi, J. Contr. Release 2002; 80:309, Drummond, Biochem. Biophys. 2000; 1463:383). The pH-sensitive proteinaceous channels of the present invention may provide release rates of drugs that are instant, i.e., within a few seconds. For example, in the lungs the pH of the airway surface liquid is reduced in subjects with inherited and acquired diseases such as cystic fibrosis and asthma as a result of lung obstruction, infection and inflammation (Coakley, J. Pancreas 2001; 2:294). Since not all lobes of the lung are affected at the same time, the use of lipid vesicles including pH-sensitive drug release channels may improve the therapeutic index of a drug administered by inhalation, wherein pathophysiological changes of the airway surface liquid, such as pH, may be used to improve inhalation therapy have not been exploited before.

Since cellular uptake of liposomes generally follows an endocytotic pathway, pH-sensitivity may also have a potential application in the delivery of drugs and genes from the endosomes into the cytosol of specific cells (Straubinger, Methods Enzymol 1993; 221:361).

In yet another embodiment, the signal may comprise an altered pH, wherein the pH is equal to or less than 6.5 pH-sensitive formulations of the invention may also be used for the release of orally taken drugs in the gastrointestinal tract. The high acid, i.e., less than pH 2, content of the stomach is neutralized in the first segment of the small intestine by pancreatic fluid. Subsequently, along the small and large intestines, the pH changes to pH 6.4 in the caecum, and again changes to neutral pH at the end of the intestines. To delay the release of drugs in the gastrointestinal tract, dosage forms have been designed which dissolve at pH 7 or above (Friend, Aliment Pharmacol Ther 1998; 12:591). However, to treat active colitis, which is characterized by a low pH at the site of inflammation, a drug should pass through the acidic environment of the stomach and remain inactive, and be subsequently activated at the site with the appropriate pH. For this purpose, the liposomes, or lipid vesicles, with pH-sensitive channels produced using methods of the present invention may be covered with a coating that is resistant to activation in the stomach, but is effectively degraded by the enzymes in the small intestines, such as enzymes that degrade several disaccharides.

In one embodiment of the present invention, liposomes containing osmo-sensitive protein channels may be used for osmo-induced drug release. Osmotic sensitive liposomes may be used to release drugs in the small intestine from stomach-resistant capsules.

Alternatively, liposomes containing light-sensitive protein channels may be used for light-induced drug release. The light-induced drug release may be useful for patient-controlled drug therapies such as analgesia for pain treatment and insulin for the treatment of diabetes. At the moment, only invasive patient-controlled systems are available for these purposes.

In one aspect, the invention provides a lipid vesicle produced by a method of the present invention, wherein the lipid vesicle comprises a biologically active molecule. In another aspect, the invention provides a composition for making a small hydrophilic molecule biologically available, wherein the composition comprises a lipid vesicle produced by a method of the present invention. The composition may be formulated and prepared for human use.

The invention further provides a composition comprising a lipid vesicle which includes a proteinaceous channel and a small hydrophilic molecule, wherein the lipid vesicle and/or the proteinaceous channel are formulated such that the proteinaceous channel is in the open state in the vicinity of or near a target cell. The open state may be achieved by supplying the proteinaceous channel in the open state, by enabling the opening of the channel when the lipid vesicle is in the vicinity of or near the target cell, and/or by providing a signal that enables opening of the channel. The proteinaceous channel may comprise an MscL or functional part, derivative and/or analogue thereof.

In one aspect, the present invention provides a composition comprising a lipid vesicle including an MscL or functional part, derivative and/or analogue thereof, wherein the composition is formulated and prepared for use in a human subject. The lipid vesicle comprises a small hydrophilic molecule capable of passing through an activated MscL. The composition may also be used in the preparation of a medicament, wherein the small molecule of the composition is intended to be delivered to the outside of a cell in a tissue.

The MscL of the present invention may be a mutant MscL or a functional part, derivative and/or analogue thereof. A functional part of MscL comprises at least the region of the E. coli MscL including residues 4 to 110 (Blount et al.). It is also possible to generate MscL proteins that comprise amino-acid substitutions, insertions and/or deletions when compared to the MscL protein found in bacteria. Such MscL mutants may also be used for the present invention provided that the MscL mutant is functional, i.e., comprises the channel activity in kind, not necessarily in amount.

The channel activity may, as will be apparent from the description, be activated by means other than pressure. With “activity in kind,” it is not meant to mean the type of triggering of the channel, but rather the channeling activity, or the capability of the channel to allow passage of a hydrophilic substance from one side of the lipid obstruction to the other. The amount of activity, both in the amount of small molecules that may pass per unit of time and the size of the pore through which the small molecule can pass, may differ. A derivative of MscL is an MscL that comprises, more or less, different modifications, i.e., post-translational, as compared to the native MscL protein. Other mutant or derivative channels may comprise MscL with genetically engineered changes in the outside loop of the protein, like receptor-recognizing domains (e.g., RGD) that, upon binding with the receptor, undergo conformational changes that induce opening of the channel. One may also add an antibody, or fragments thereof, to the loop of the protein that induces channel opening after ligand binding. An MscL analogue is a molecule comprising the same activity in kind which allows passage of hydrophilic molecules through a lipid obstruction other than native MscL, not necessarily in amount.

In another aspect, the invention provides a method of generating a vehicle for delivery of a small hydrophilic molecule to a cell, wherein the method comprises generating a lipid vesicle including a proteinaceous channel in an aqueous fluid, wherein the vehicle is formulated such that the proteinaceous channel is in the open state in the vicinity of or near the cell. The proteinaceous channel of the vehicle assumes the open state upon entering the vicinity of or being near the cell. The lipid vesicle further comprises the small molecule. A method for generating the vehicle described herein may also be used to generate a composition.

In one embodiment, a lipid vesicle of the invention further comprises a non-channel protein. The non-channel protein is a binding molecule capable of binding to a binding partner in the tissue, thus enabling, at least, a prolonged stay of the vesicle in the tissue and/or near a target cell.

In another aspect, the invention provides the administration of a lipid vesicle comprising an MscL for controlling delivery of a small hydrophilic molecule to a target tissue in a subject.

A lipid vesicle of the present invention may be used to deliver a small molecule to any part of the body of a subject. For instance, the lipid vesicle may be used to deliver a small molecule to tissues with a permeable endothelium such as the liver, the spleen, areas of inflammation or tumor-bearing tissues.

A lipid vesicle of the present invention may comprise lipids, but may also comprise other types of molecules. For instance, glycolipids or other lipids that are modified in ways that maintain the classical bipolarity of a lipid molecule in kind, not necessarily in amount, are also referred to as lipids in the present invention.

In one embodiment of the invention, the lipid vesicle comprises a liposome, such as a long circulating liposome. Long circulating liposomes are typically small, i.e., 150 nm or smaller, neutral and have a specific composition, such as cholesterol-containing with either phosphatidylcholine and PEG or sphingomyelin, etc.

Since MscL will typically be a protein foreign to the subject, it is conceivable that, upon repeated administration, an immune response may be mounted by the host or subject. To allow at least a partial evasion of the immune system of the host, so-called masking groups may be attached to the outside of the lipid vesicle. One example of a masking group may comprise PEG.

In yet another embodiment, the invention provides a use of a lipid vesicle or a composition according to the present invention for delivering a small hydrophilic molecule to a biological system for a non-medical purpose. The invention further provides the use of a lipid vesicle or a composition of the invention for the preparation of a medicament. Further provided is the use of a mechanosensitive channel for modulating the bio-availability of a small hydrophilic molecule packaged in a lipid vesicle.

### BRIEF DESCRIPTION OF THE DRAWINGS

FIG. 1. SDS-PAGE stained with Coomassie Brilliant Blue (molecular weight markers indicated in kDa on the left of lane A, and purified detergent-solubilized MscL in lane B), and a Western blot (lane C).

FIG. 2. Electrospray ionization mass spectrometry of G22C-MscL-6His and its MTSES conjugate. Solid line represents spectrum of G22C-MscL-6His. Not all peptides that are present in the sample are indicated in the spectrum. Broken line represents spectrum of MTSES conjugated with G22C-MscL-6His. The masses are indicated at the peaks and show that all proteins are conjugated.

FIG. 3. Equilibrium centrifugation of sucrose gradients of proteoliposomes. 6His-MscL purified with Triton X-100 and incorporated in liposomes is titrated with 4.0 mM Triton X-100, Rsat, is represented with open squares, while liposomes titrated with 10.0 mM Triton X-100, Rsol, is represented with closed squares. After centrifugation, the gradients were fractionated (0.5 mL) and assayed for the presence of lipids and protein. All protein, as determined by Western blotting as shown in the inset, is shown to be associated with the lipids as determined by measuring fluorescence (AU) of R₁₈.

FIG. 4. Freeze-fracture image of proteoliposome showing the MscL channel protein as a transmembrane vesicle (white box).

FIG. 5. Patch-clamp recordings of channel activities at −20 mV from MscL reconstituted into liposomes of different lipid compositions as indicated. Pressure in the pipette, relative to atmospheric, is shown in the lower traces, and recording of the current through a patch of membrane excised from a blister is shown in the upper traces.

FIG. 6. Pressure dependence of the MscL channel reconstituted in liposomes of different lipid composition. Open probability in the patch of a membrane with a lipid composition of PC: PS, 90:10 m/m (A) and PC: PE, 70:30, m/m (B) versus the applied pressure. Smooth curves are Boltzman fits.

FIG. 7. Calcein efflux from liposomes with MscL (closed circles) and without MscL (closed squares) are shown as a function of a decrease in Osmolality of the external medium. A small volume (typically 20 μL) containing proteoliposomes in iso-osmotic buffer is rapidly diluted with buffer of decreasing osmolality and calcein release was determined by dividing the fluorescence at 100 sec after dilution by the total fluorescence obtained after Triton X-100 lysis.

FIG. 8. Calcein release under iso-osmotic condition mediated by conjugated G22C-MscL-6His. Calcein release of MTSES conjugated G22C-MscL-6His channel protein reconstituted into liposomes (PC:Chol, 60:40, m/m) (closed circles). Liposomes with the same lipid composition and sample treatment as above but without MscL (closed squares).

FIG. 9. Effect of 5 mol % DGPE-PEG (2000) on the calcein release from liposomes (PC:Chol, 60:40, m/m). Calcein release from PC:Chol:DGPE-PEG (2000) liposomes in the presence of buffer (closed triangles), rat plasma (closed circles), and human plasma (closed squares). Calcein release from liposomes without DGPE-PEG (2000) (closed diamond).

FIGS. 10A-10D. Patch clamp recordings of MscL channel activities at +20 mV in spheroplasts. Pressure in the pipette, relative to atmospheric, is shown in the lower traces of FIGS. 10A and 10B, and recordings of the current through a cell attached patch of a spheroplast are shown in the upper traces of FIGS. 10A and 10B. FIG. 10A shows results of MscL mutant G22C in spheroplast before MTSET attachment, and FIG. 10B is the same as FIG. 10A but after MTSET attachment. Buffer: 200 mM KCl, 90 mM MgCl₂, 110 mM CaCl₂, 5 mM HEPES, pH 6.0. FIGS. 10C and 10D show the histograms of the conductivity preferences of FIGS. 10A and 10B, respectively.

FIG. 11. Calcein efflux from liposomes (DOPC:DOPS, 90:10, mol/mol) with MscL mutant G22C (protein to lipid, 1:20 wt/wt). 1 mM MTSET, 2.5 mM MTSEA, and 10 mM MTSES was added at the time indicated by the arrow.

FIG. 12. Calcein efflux from liposomes (DOPC:Cholesterol, 80:20, mol/mol) with MscL mutant G22C (closed squares) and without MscL mutant G22C (closed circles).

FIG. 13. ESI-MS analysis of the IMI conjugation to a single cysteine mutant of MscL at position 22. Unconjugated G22C-MscL with an expected mass of 15,697 Da (closed squares). IMI conjugated G22C-MscL with a 156 Da mass increase (closed triangles).

FIGS. 14A-14D. Patch clamp recordings of imidazole coupled and uncoupled MscL mutant G22C channels arc shown in FIG. 14B and FIG. 14A, respectively. 5 μl of G22C spheroplasts were incubated with IMI (2 mM final concentration) or with patch buffer overnight at 4° C. The next day, currents through cell-attached patches held at +20 mV were recorded for unlabeled (FIG. 14A) and labeled (FIG. 14B) proteins and histograms showing the conductance states of each recording are illustrated in FIG. 14C and FIG. 14D, respectively.

FIG. 15. Different pKa's of substituents for MscL mutant G22C labels.

FIGS. 16A-16H. Patch clamp recordings of patches excised from proteoliposomes containing BP coupled and uncoupled MscL mutant G22C channels. FIG. 16A shows the labeled channel behavior at pH 7.2 and the histogram showing the conductance levels is given in FIG. 16B. FIGS. 16C and 16E show the behavior of unlabeled MscL channel at pH 5.2, respectively. FIGS. 16D and 16F show the labeled channels at pH 5.2, and their conductance histograms are given in FIG. 16G for the unlabeled and FIG. 16H for the BP labeled MscL channel, respectively. Measurements were performed with +20 mV constant voltage.

FIG. 17. Structure of DTCP1 in the open state (A) and in the closed state (B). The molecule can reversibly isomerize depending on the wavelength of the absorbed light.

FIG. 18. ESI-MS analysis of the DTCP1 conjugation to a single cysteine mutant of MscL at position 22. Unconjugated G22C-MscL with expected mass of 15,697 Da (closed squares). DTCP1 conjugated G22C-MscL with a 344 Da mass increase (closed triangles).

FIG. 19. Absorption spectra of DTCP1. Open isomer A has a maximum at 260 nm and no absorbance at wavelengths higher than 400 nm; closed isomer B has a very distinct peak with a maximum at 535 nm. Gray line shows substracted spectra of open and closed isomer.

FIG. 20. Substracted spectra of open and closed isomer of DTCP1 after conjugation to G22C-MscL and reconstitution in DOPC:DOPS (90:10, mol/mol) lipid bilayer.

FIG. 21. Four switching cycles of DTCP 1 conjugated to MscL and reconstituted in lipid bilayer.

FIG. 22. Photochromic molecule SP1 in its spiropyran form (left) and merocyanine zwitterionic form (right).

FIG. 23. Absorption spectrum of SP1 conjugated to MscL in spiropyran form SP1 and after irradiation in highly charged merocyanine form MC1.

FIG. 24. Reversible switching between spiropyran (SP) and merocyanine (MC) form by alternating irradiation with UV and visible light.

FIG. 25. Structure of sodium di(C4azobenzene-O-C6)-phosphate in the trans and cis conformation.

FIG. 26. UV/Vis spectra of sodium di(C4azobenzene-O-C6)-phosphate (mol. 7) in the trans and cis state. The molar ratio of DSP to sodium di(C4azobenzene-O-C6)-phosphate is 95:5. Concentration of sodium di(C4azobenzene-O-C6)-phosphate is 12.5 μM.

FIG. 27. Repeated cycles of the isomerization of lipid 6 in a vesicle which is composed of 95% DOPC and 5% lipid (mol. 6). For the trans conformation, the absorbance at 349 nm is given and for the cis conformation the absorbance at 313 nm is given.

FIG. 28. UV/Vis spectra of lipid (mol. 6) in a vesicle which is composed of 95% DOPC and 5% lipid (mol. 6). The times indicated are the irradiation times. The sample was irradiated with 365 nm light.

FIG. 29. DSC graphs of pure DSP and a mixture of DSP and sodium di(azobenzene-O-C6)-phosphate (molar ratio 95:5).

FIG. 30. Urinary excretion of MAG3 after subcutaneous or intravenous injection. Free MAG3 injected intravenously (open circle, right y-axis), free MAG3 injected subcutaneously (closed circle), MAG3 in “empty” liposomes injected subcutaneously (open squares), MAG3 in G22C-MscL-containing liposomes injected subcutaneously (closed squares).

FIG. 31. Subcutaneous pH-reduction. MES buffer (0.5 ml, pH 6.1) of different molarities was injected subcutaneously in conscious rats.

FIG. 32. Urinary excretion of IOT after subcutaneous injection. Free IOT (open squares), IOT in DOPC/PS liposomes (closed squares), IOT in DOPC/PE liposomes (closed triangles).

FIG. 33. SDS-PAGE gel stained with Coomassie Brilliant Blue. Lane A: vesicles containing the overexpressed protein, lane B: molecular weight marker, and lane C: purified protein.

FIG. 34. Freeze-fracture image of proteoliposome showing the MscL channel protein as a transmembrane particle (white box).

FIG. 35. A typical trace of channel activity of MscL^{Ll} in MscL^{Ec-}/MscS^{Ec+} E. coli cells. The upper trace shows the current across the membrane due to channel activity. Flow of current is shown upward in all traces. From left to right, in time, the first two channels of small conductivity open (also shown in enlarged left panel) and, later, the opening of a single MscL^{Ll} are shown (also shown in enlarged left panel). The lower trace indicates the pressure applied to the membrane. The panels show enlargements of the upper trace.

FIG. 36. Top panel shows the dependence of opening chance of the MsCL^{Ec} channel on the applied pressure in the pipette. The sigmoidal curve shows that no channels open at 0 mmHg pressure and that all channels are open at 90 mmHg. The center panel shows the time channels spend in the open state. The bars indicate the distribution of opening times of the L. lactis MscL (<0.1 ms and 0.7 ms). Resolution of the traces does not allow analysis on a shorter time scale. Bottom panel shows the relationship between voltage and current through the channel. The slope of the graph is the conductivity of the channel, which is 2.5 nS.

FIG. 37. Electrophysiological analysis of MscL^{Ll} in left panel, PC:Cholesterol 8:2 mol/mol, and right panel, PC:PS 9:1. Protein:lipid ratio in both cases is 1:1000.

FIG. 38. Calcein release from PC:PS (9:1) proteoliposomes containing MscL^{Ll} (protein:lipid 1:500) or not containing any protein after dilution of the isoosmotic buffer, with dH₂O to indicated dilutions. As illustrated, the protein-containing liposomes release more calcein than the liposomes without protein. This is MscL^{Ll} mediated efflux of the calcein.

FIG. 39. Efflux of FITC-insulin under different conditions from DOPC:DOPS (9:1, mol/mol) liposomes containing MscL mutant G22C. Not filtered (200 μl proteoliposomes); not filtered after triton (200 μl proteoliposomes with Triton X-100); filtered (200 μl after filtration); 5′ or 10′+MTSET (200 μl proteoliposomes incubated with 1 mM MTSET for 5′ or followed by filtration; 10′-MTSET (200 μl proteoliposomes incubated 10′ without MTSET followed by filtration; and Triton (200 μl proteoliposomes with Triton X-100 followed by filtration).

FIG. 40. Dependence of the amount of membrane tension needed to open the MscL channel on the lipid composition of the membrane. Increase in DOPE content results in a decrease in the membrane tension needed to open the channel.

### DETAILED DESCRIPTION OF THE INVENTION

Non-limiting examples of small molecules that may advantageously be used in a lipid vesicle of the invention include:

Interleukins: peptides and proteins that modulate the immune response;

Diphtheria toxin and fragments thereof: potent inhibitor of protein synthesis in human cells;

Muramyl dipeptide: activator of immune system; macrophage-mediated destruction of tumor cells;

Cis-4-hydroxyproline: potential treatment for lung fibrosis;

Cisplatin and derivatives thereof: cancer treatment;

Cytosine arabinose: cancer treatment;

Phosphonopeptides: antibacterial agent;

β-Glucuronidase: activator of prodrugs (e.g., epirubicin-glucuronide);

Cytostatic drugs, e.g. doxorubicin, ciplatin etc.; and

Small therapeutic proteins/peptides, e.g., interleukins, insulin, growth factors, chemokines.

Bio-availability of small molecules can be achieved in various ways. Typically, the small molecule is administered to the biological system where it is to be made available. However, the reverse may also be true, in that the small molecule is first provided. In this situation the biological system is provided later. The purpose of the latter situation may be to, at least in part, prevent further development of the biological system, such as in a decontamination setting.

As used herein, the phrase “altering the open-probability of a proteinaceous channel” will be used to refer to the shifting of the equilibrium of the open/closed state of the proteinaceous channel such that the equilibrium lies more to the open state or more to the closed state at the conditions used.

### EXAMPLES

### Example 1

Material and Methods

MscL Expression and Purification.

E. coli PB 104 cells containing the plasmid pB 104 which carries the MscL-6His construct were grown to mid-logarithmic phase in Luria Bertani (LB) medium in a 1 OL fermentor and induced for 4 h with 0.8 mM IPTG (Blount et al.). The cells were French-pressed and membranes were isolated by differential centrifugation, as previously described (Arkin et al.). The membrane pellet (5-8 g wet weight) was solubilized in 100 mL of buffer A (50 mM Na₂HPO₄.NaH₂PO₄, 300 mM NaCl, 10 mM imidazole) containing 3% n-octyl β-glucoside. The extract was cleated by centrifugation at 120,000×g for 35 min., mixed with 4 mL (bed volume) Ni²⁺-NTA agarose beads (Qiagen, Chatsworth, Calif.), equilibrated with buffer A and gently rotated for 15 min., i.e., batch loading. The column material was poured into a Bio-Spin column (Bio-Rad) and washed with 10 column volumes of buffer B (same as buffer A, except 1% n-octyl 13-glucoside is added) followed by 5 column volumes of the buffer B, but with the addition of 100 mM imidazole. The protein was eluted with buffer B which further included 300 mM imidazole. Eluted protein samples were analyzed by fractionation on an SDS-15% polyacrylamide gel followed by staining with Coomassie Blue or transferring the fractionated proteins to PVDF membranes by semi-dry electrophoretic blotting for immunodetection with an anti-His antibody (Amerham Pharmacia Biotech). Immunodetection was performed with an alkaline phosphatase conjugated secondary antibody as recommended by the manufacturer (Sigma).

Electrospray Ionization Mass Spectrometry of Detergent Solubilized MscL proteins.

Purified, detergent solubilized G22C-MscL-6His was heated to 60° C. for 15 min. and precipitated protein was spun down at 14,000 rpm in a tabletop centrifuge (Eppendorf) for 5 min. The pellet was dissolved in 50% formic acid and 50% acetonitril prior to electrospray ionization mass spectrometry (ESI-MS) analysis. The average molecular masses of the proteins were calculated from the m/z peaks in the charge distribution profiles of the multiple charged ions. Spectral deconvolution was performed on the peaks over the mass range from 800 to 1700 using the computer program MacSpec (Sciex). All molecular masses quoted herein are average, chemical atomic masses.

2-Sulfonatoethyl methanethiosulfonate labeling of G22C-MscL-6His.

The single cysteine mutant, G22C-MscL-6His, was labeled with (2-sulfonatoethyl)methanethiosulfonate (MTSES). A suspension of 20-30 μM of G22C-MscL-6His in buffer B with 300 mM imidazole (0.5 mL final volume) was incubated with 0.6 mM MTSES at 4° C. for 30 min. Conjugation was monitored using ESI-MS.

Membrane Reconstitution of 6His-MscL.

Dry lipid mixtures were prepared by co-dissolving lipids (Avanti Polar Lipids, Alabaster, Ala.) in chloroform, in weight-fractions as indicated in the experiments, and removing the chloroform by evaporation under vacuum for 4 h. All acyl chains of the synthetic lipids were of the type dioleoyl, unless indicated otherwise. The dried lipid film was dissolved (20 mg/mL) in 50 mM potassium phosphate, pH 7.0, followed by three freeze/thaw cycles. An aliquot, 200 μL of the rehydrated liposomes and 5% n-octyl P-glucoside, was added to 200 μL purified 6His-MscL. Final protein-to-lipid molar ratio was determined as indicated in the experiments. Subsequent membrane reconstitution was achieved by exhaustive dialysis into a buffer containing 0.1 mM Na₂HPO₄.NaH₂PO₄ at pH 6.8 that contained detergent-absorbing Bio-Beads SM-2 (Bio-Rad, Inc.).

Sucrose Gradient Centrifugation.

Discontinuous sucrose gradients were employed to analyze membrane reconstituted 6His-MscL as described (Knol et al.).

Freeze-Fracture Electron Microscopy.

Freeze-fracture electron microscopy of membrane-reconstituted 6His-MscL was performed as described (7).

Electrophysiologic Characterization of Membrane-Reconstituted MscL.

MscL was reconstituted into liposomes of different lipid composition and aliquots of 200 μL were centrifuged at 48,000 rpm in a tabletop ultracentrifuge (Beckmann). Pelleted proteoliposomes were resuspended into 40 μL buffer C (10 mM 4-morpholinepropanesulfonic acid (MOPS)-buffer, 5% ethylene glycol, pH 7.2), and 20 μL droplets of the unsuspended proteoliposomes were subjected to a dehydration-rehydration cycle on glass slides (Delcour et al.). Rehydrated proteoliposomes were analyzed employing patch-clamp experiments as described (Blount et al.).

In vitro Release Profiles of a Model Drug from Proteoliposomes.

The percentage release of a fluorescent model drug, calcein, from MscL-containing liposomes was calculated from the dequenching of calcein fluorescence using the following equation:

$$\% Release = \frac{{F}_{x} - {F}_{0}}{{F}_{t} - {F}_{0}} \times 100$$

Where F₀ is the fluorescence intensity at zero time incubation, Fₓ is the fluorescence at the given incubation time-points and Ft is the total fluorescence obtained after Triton X-100 lysis. Fluorescence was monitored with an SLM 500 spectrofluorimeter in a thermostatted cuvette (1 mL) at 37° C., under constant stirring. Excitation and emission wavelengths were, respectively, 490 (slit 2 nm) and 520 nm (slit 4 run). The experiments were performed at lipid concentrations of approximately 50 μM. Control and MscL-containing liposomes were prepared as described followed by mixing with an equal volume of 200 mM calcein in PBS buffer. A freeze-thaw cycle was repeated three times followed by extrusion through a 100 nm polycarbonate membrane (Mayer et al.). The liposomes were separated from free calcein with a Sephadex 50 column chromatography equilibrated with PBS (160 mM NaCl, 3.2 mM KCl, 1.8 mM KH₂PO₄, 0.12 mM Na₂HPO₄, 1.2 mM EGTA, pH 8.0) which was isotonic to the calcein-containing buffer.

Results and discussion

Overexpression and purification of the MscL channel protein.

Since the expression level of His-MscL in E. coli was relatively low, based on the absence of a significant IPTG-inducible band on an SDS-PAGE, attention was focused on obtaining a high biomass during fermentation and a high yield after protein purification.

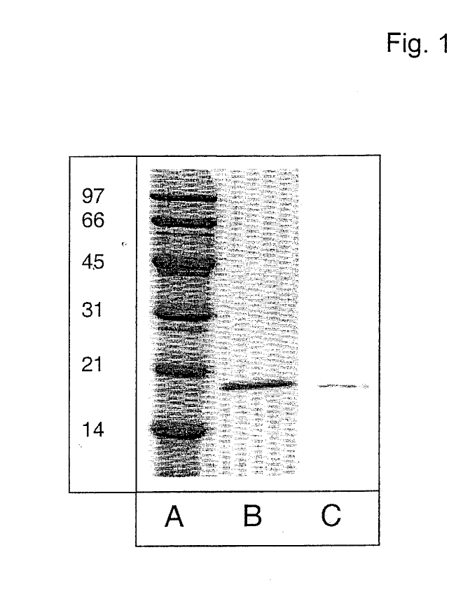

The His-tagged MscL could be purified to apparent homogeneity in a single step using nickel chelate affinity chromatography as shown by SDS-PAGE (FIG. 1, lane B). The yield of the eluted His-tagged MscL was ±2 mg per liter of cell-culture with an estimated purity of >98% based on analysis using SDS-PAGE and Coomassie Brilliant Blue staining.

The rate of excretion via MscL of small molecules is >10,000 mmol/sec.×mg of cell protein, i.e., when the protein is in the open state. Since the expression level of MscL in wild-type bacteria is 4-10 functional units per cell and the MscL channel is a homopentamer of 15,000 Da, it is estimated that the flux via a functional MscL channel is >10⁶×s⁻¹. This activity of MscL is such that, on average, 5 molecules of pentameric MscL per liposome with a diameter of 400 nm should suffice. Such a liposome contains approximately 1.67×10⁶ molecules of lipid, wherein the molar ratio of lipid over MscL will be 0.67×10⁵. Consequently, 2 mg of MscL will yield 6 g of proteoliposomes.

Electrospray Ionization Mass Spectrometry of Detergent Solubilized MscL proteins.

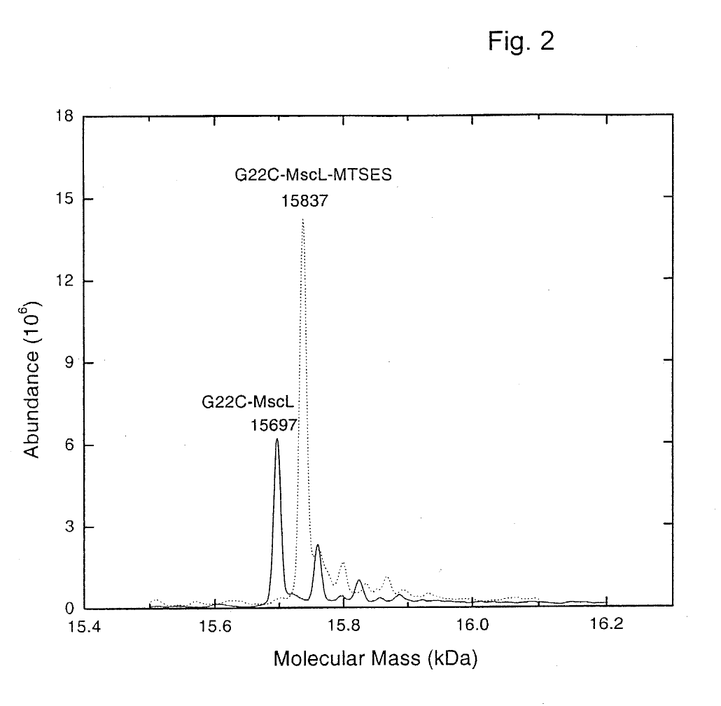

ESI-MS is an accurate and effective method to verify primary sequences of the 6His-MscL protein and the stoichiometry of conjugation reactions. FIG. 2 shows the ESI-MS spectra of the G22C-MscL-6His and the MTSES conjugated G22C-MscL-6His samples.

Based on the deduced amino acids, the average molecular weight of G22C-MscL-6His is 15,826 Da. ESI-MS analysis of G22C-MscL-6His resulted in a molecular weight of 15,697 Da, which corresponds to the deduced molecular weight minus a methionine. This observation would be consistent with an excision of the N-terminal methionine as reported for many proteins expressed in E. coli (Hirel et al.). ESI-MS analysis of the MTSES conjugated G22C-MscL-6His resulted in a molecular weight of 15,837 Da, which corresponds with the calculated mass increase of the MTSES conjugation. ESI-MS analysis is used herein to verify the average masses of MscL mutants and the products of conjugation reactions.

Membrane reconstitution into liposomes of different lipid compositions.

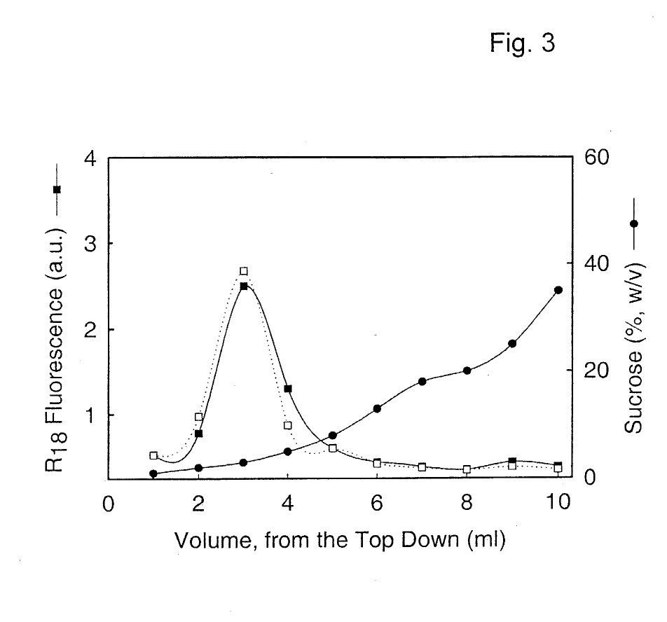

Purified detergent-solubilized MscL was reconstituted into preformed liposomes, which were titrated with low amounts of detergent. After removal of the detergent by adsorption onto polystyrene beads, proteoliposomes were formed. The proteoliposomes were characterized by equilibrium sedimentation on a sucrose gradient as shown in FIG. 3. All 6His-MscL protein detected by the Western blot (inset in FIG. 3) was associated with the lipid bilayer as detected by octadecylrhodamine-β-chloride (R₁₈) fluorescence.

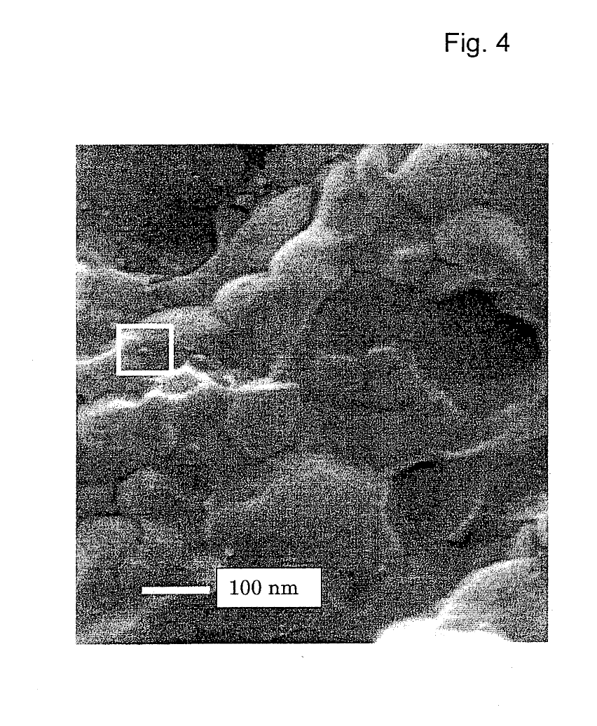

Association of the 6His-MscL protein with the liposomes does not necessarily mean the protein is inserted correctly into the lipid bilayer. Correctly inserted MscL protein should be a transmembrane protein and show up as an intra-membrane vesicle (IMP) in a freeze-fracture image as shown in the white boxed area of FIG. 4.

The equilibrium sedimentation and freeze-fracture electron microscopy experiments provided structural evidence for the correct reconstitution of the 6His-MscL protein into lipid bilayers.

Electrophysiologic characterization of membrane-reconstituted MscL activity.

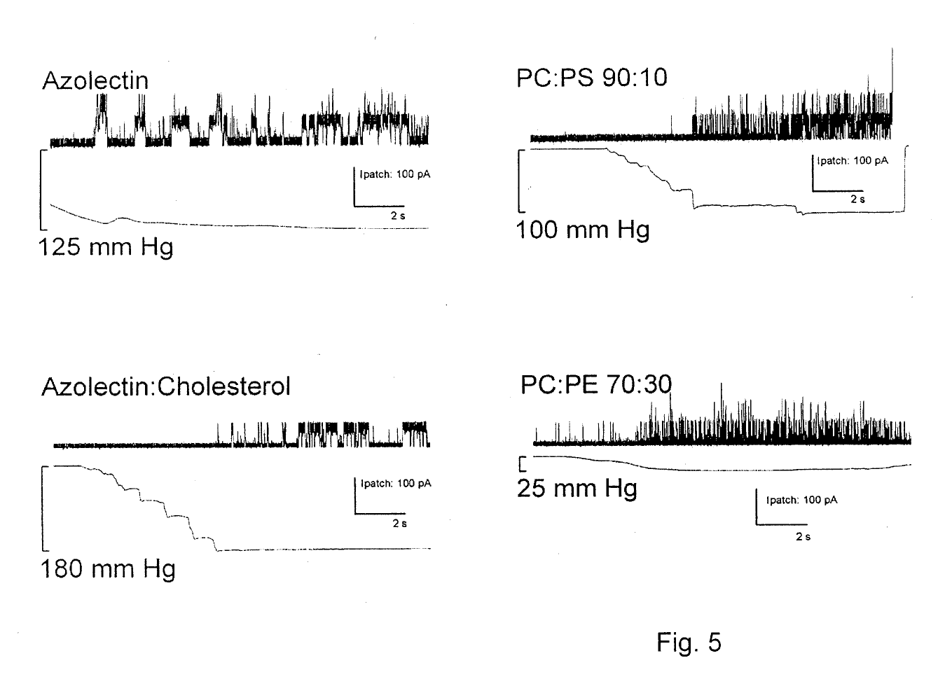

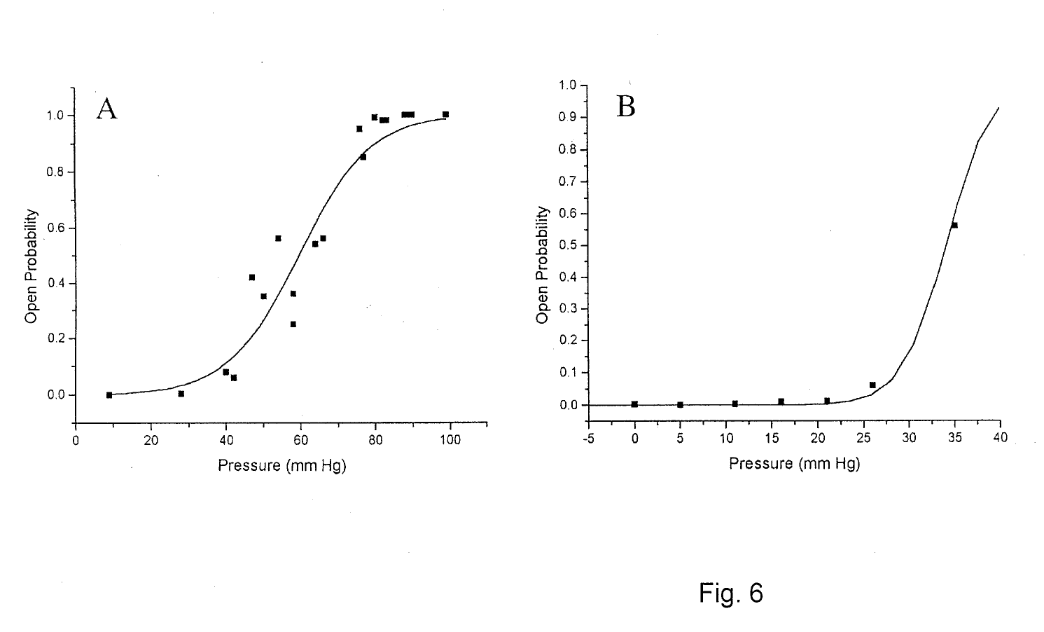

The purified protein reconstituted into phospholipid liposomes forms functional mechanosensitive channels, as seen from the traces in FIG. 5 at different pipette pressures (mechanical activation). The MscL open probability plotted against pressure can be fitted with a Boltzman distribution (FIG. 6).

Reconstituted MscL is active in the absence of negatively charged lipid headgroups (FIG. 5, PC:PE 70:30). This is a very important finding since negatively charged headgroups prevent targeting to most target sites in the human body. Additionally, these experiments show that the pressure threshold is significantly affected by the lipid composition of the membrane-reconstituted MscL channels (FIG. 6). This allows tailor making of the drug release profiles of the MscL channel to the specific needs. For example, several 6His-MscL mutants with altered gating properties, mutants that are hypersensitive to membrane tension and mutants with increased open probability at lower pH-values are known.

In vitro release profiles of a model drug.

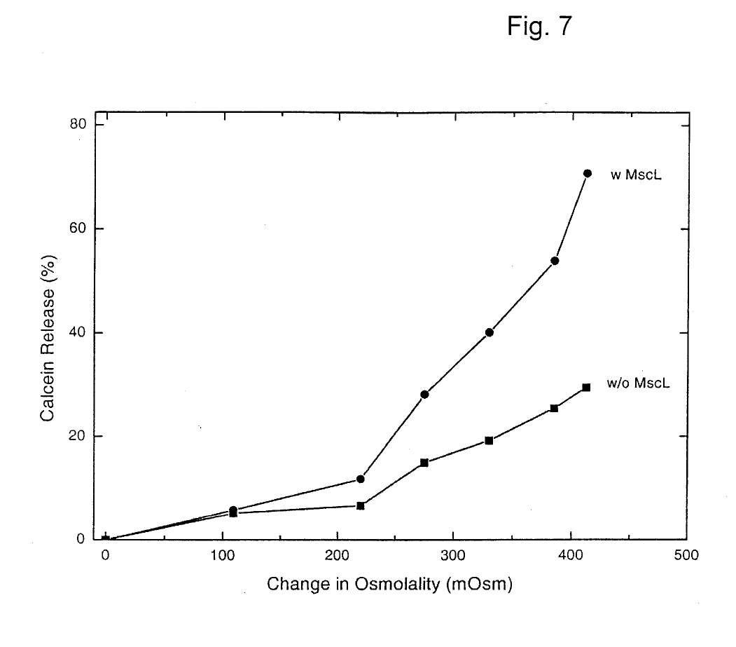

The fluorescence efflux-assay was developed to monitor the MscL-mediated release profiles. Liposomes (DOPC:Chol, 60:40, m/m) with and without MscL-6His were subjected to an osmotic downshock, thus effectively increasing the membrane tension, to monitor the calcein release. As shown in FIG. 7, less calcein remained in the liposomes containing MscL (closed circles) relative to the liposomes without MscL (closed squares) when change in osmolality was larger than 200 mOsm. This data demonstrates that, upon osmotic downshock, liposomes containing reconstituted MscL exhibit a greater efflux of calcein than liposomes without MscL. This MscL-mediated efflux is consistent with the electro-physiologic analysis, showing that the MscL is reconstituted into membranes of synthetic lipids while retaining its functional properties.

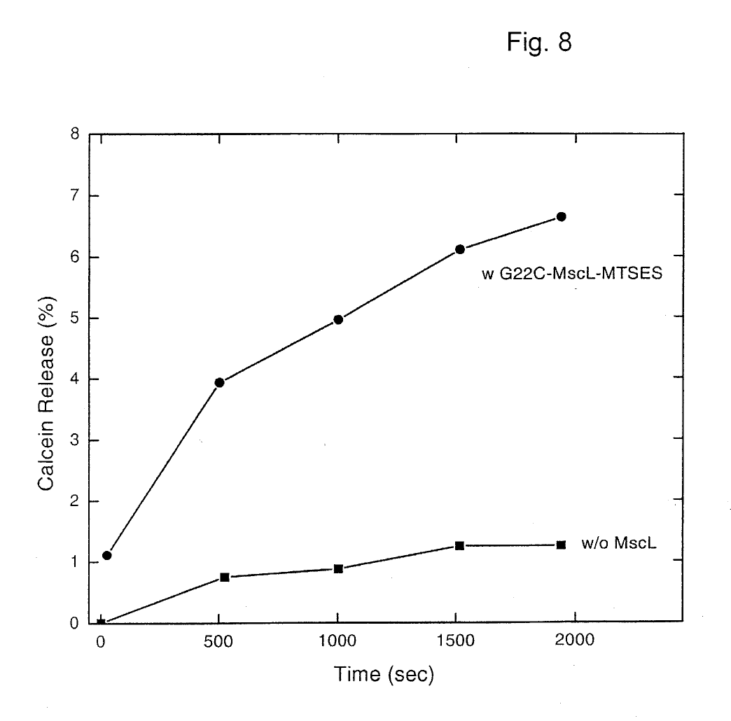

For controlled release of drugs at the target site, membrane tension may not be the most promising stimulus since little is known about osmotic differences in the human body. Several alternatives to activate the MscL channel at the target site are described. Introducing a charge through conjugation of MTSES to cysteine at position 22 serves as an example for channel activation under iso-osmotic conditions. ESI-MS analysis of the MTSES conjugated G22C-MscL-6His protein showed that MscL monomers were conjugated to a stoichiometry of 1:1. The conjugated G22C-MscL-6His samples were subsequently reconstituted into liposomes as described above and calcein release was measured as shown in FIG. 8. This data demonstrates that, in the absence of an increased membrane tension, MscL exhibits drug release from drug laden synthetic liposomes. MscL conjugates described herein will release drugs at the target site as a function of pH, light activation and specific interactions with target associated molecules.

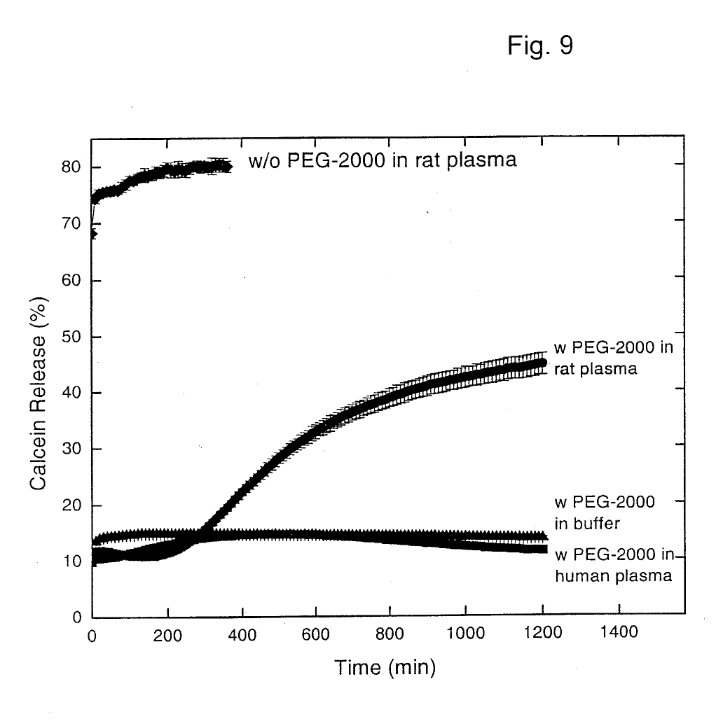

The integrity of liposomes including phosphatidylcholine is affected at first contact with a biological milieu following intravenous injection (Damen et al.). The integrity of liposomes (PC:Chol:DGPE-PEG, 55:40:5, m/m/m) was studied as a function of the molecular mass of the PEG group attached to the DGPE lipid in the presence of rat and human plasma at 37° C. Calcein release from the liposomes without DGPE-PEG and with 5 mol % DGPE-PEG (2000) are shown in FIG. 9. This data shows that the addition of 5 mol % of DGPE-PEG (2000) significantly increases liposomal integrity in rat and human plasma for up to several hours and serves to prevent drug leakage during the travelling time to the target cells.

### Example 2

Delivery of a substance from liposomes through a charge-induced channel opening.

Many substances can cause activation of the MscL channel. One example in this context is a group of compounds that is capable of associating with MscL mutant G22C (Yoshimura et al.). Attachment of these positively charged reagents {([2-(Trimethylammonium)ethyl]methanethiosulfonate) (MTSET) and (2-aminoethyl methanethiosulfonate) (MTSEA) or negatively charged (sodium (2-sulfonatoethyl) methane thiosulfonate) (MTSES)} to the cysteine under patch clamp causes MscL to gate spontaneously, even when no tension is applied (Yoshimura et al.). These results indicate that chemically charging the pore constriction at amino acid position 22 opens the MscL channel.

Since experiments have been performed on spheroplasts containing the MscL mutant G22C in its natural environment wherein the spheroplasts contained a wide variety of lipidic and proteinaceous molecules, it was relevant to show that the methanethiosulfonate compounds attach specifically to the MscL mutant and this charged-induced gating occurs in artificial lipid membranes without the involvement of other cellular or membrane components.

### Example 2A

Example 1, FIG. 2, shows that a methanethiosulfonate compound covalently attaches to the MscL mutant G22C in a one-to-one stoichiometry. Example 2A ishows that the effect of MTSET attachment to MscL mutant G22C on the pressure sensitivity of the channel and the change in preference for specific conductance states under patch clamp conditions.

Materials and Methods

MscL mutant G22C containing six C-terminal histidine residues was constructed using standard molecular biology techniques. Expression, purification, membrane reconstitution, and patch clamp analysis were performed as described in Example 1 or as described below.

MscL Expression and Purification. E.coli PB1O4 cells containing the plasmid pB104 carrying the MscL-6His construct were grown to early-logarithmic phase in Enriched medium (Yeast extract 150 g/l, Bactotrypton 100 g/l, NaCl 50 g/l, K₂HPO₄ 25 g/l, KH₂PO₄ 25 g/l, Antifoam A 2 ml/l; after sterilization add 1.5 g Amp, 10 ml 1000×F⁺² stock (Fe⁺² stock: 0.278 gr FeSO₄, 7H₂O in 100 ml 1N HCl) and 10 ml 1000×spore-elements stock (Spore-elements per 100 ml: EDTA 1 gr, ZnSO₄.7H₂O 29 mg, MnCl₂.4H₂O 98 mg, CoCl₂.6H₂O 254 mg, CuCl₂ 13.4 mg, CaCl₂ 147 mg, pH 4 with NaOH) in a 15L fermentor and induced for 4 h with 1.0 mM IPTG. (Blount et al.) Harvested cells were French-pressed and membranes were isolated by differential centrifugation, as previously described (Arkin et al.). The membrane pellet (2.4 g wet weight) was solubilized in 30 ml of buffer A (50 mM K₂HPO₄.KH₂PO₄ pH 8.0, 300 mM NaCl, 35 mM imidazole, pH 8.0, 3% n-octyl β-glucoside). The extract was cleared by centrifugation at 120,000×g for 35 min., mixed with 4 ml (bed volume) Ni²⁺-NTA agarose beads (Qiagen, Chatsworth, Calif.) equilibrated with wash buffer (300 mM NaCl, 50 mM K₂HPO₄.KH₂PO₄ pH 8.0, 35 mM imidazole pH 8.0, 1% n-octyl β-glucoside) and gently rotated for 30 min. at 4° C. (batch loading). The column material was poured into a Bio-Spin column (Bio-Rad) and washed with 25 ml of wash buffer, with 0.5 mL/min. flow rate. The protein was eluted with wash buffer containing 235 mM imidazole. Eluted protein samples were analyzed by fractionation on an SDS-15% polyacrylamide gel followed by staining with Coomassie Brilliant Blue or transferring the fractionated proteins to PVDF membranes by semi-dry electrophoretic blotting for immunodetection with an anti-His antibody (Amersham Pharmacia Biotech). Immunodetection was performed with an alkaline phosphatase conjugated secondary antibody as recommended by the manufacturer (Sigma).

Electrophysiologic characterization of membrane-reconstituted MscL.

MscL was reconstituted into liposomes of different lipid composition and aliquots of 200 μL were centrifuged at 70,000 rpm in a tabletop ultracentrifuge (Beckmann). Pelleted proteoliposomes were resuspended into 30 μL buffer C (10 mM 4-morpholinepropanesulfonic acid (MOPS)-buffer, 5% ethylene glycol, pH 7.2), and 15 μL droplets were subjected to a dehydration-rehydration cycle on glass slides (Delcour et al.). Rehydrated proteoliposomes were analyzed employing patch-clamp experiments as described previously (Blount et al.).

Giant spheroplasts were prepared as explained before (Blount, P. et al., Methods Enzymol. Vol. 294:458-482, 1999).

Results and Discussion

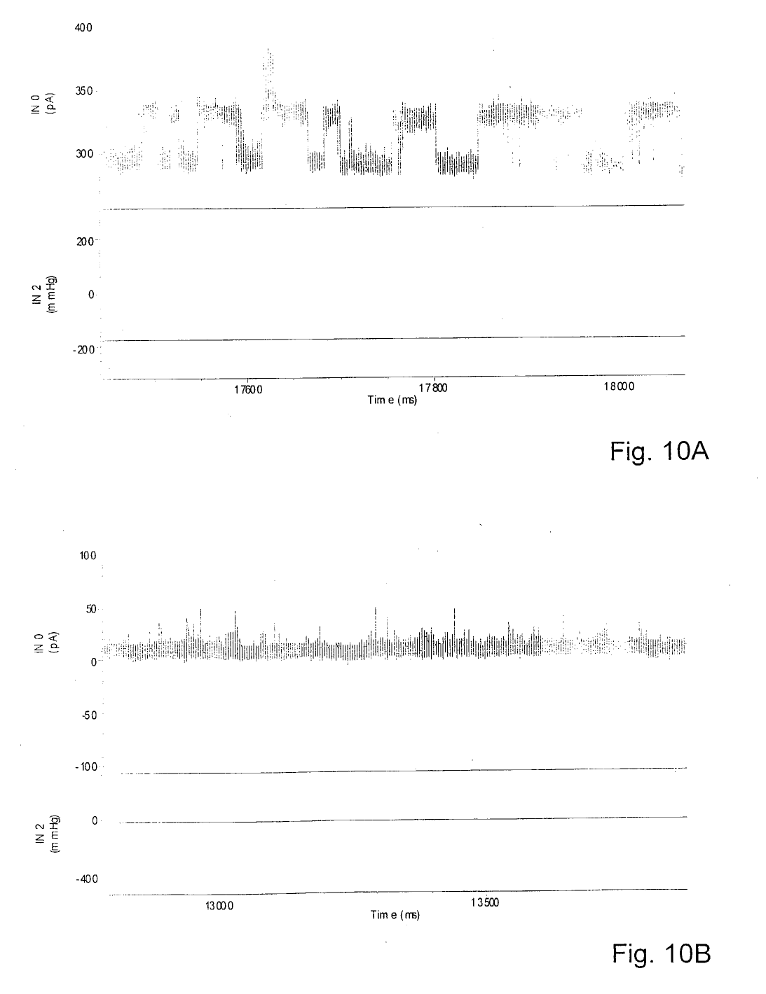

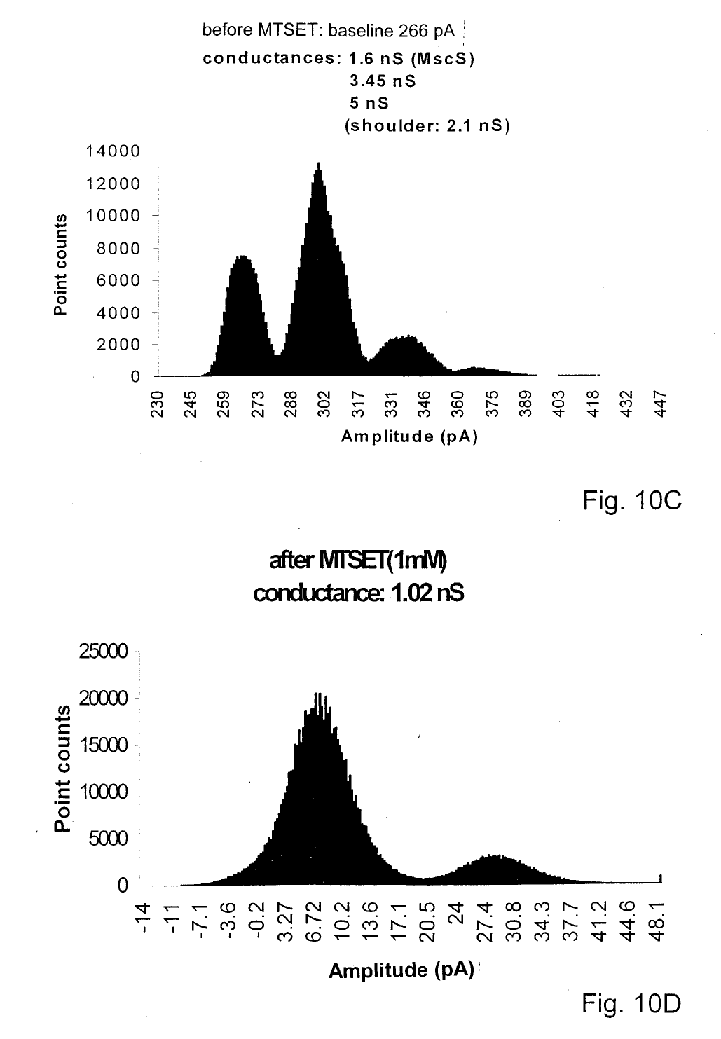

The electrophysiologic characterization of the MscL mutant G22C as shown in FIGS. 10A and 10C resulted in similar channel properties as described in the literature with respect to pressure sensitivity and a conductance of 3.42 nS. The other observed conductances are from MscS (1.6 nS), another mechanosensitive channel in spheroplast, and most probably a simultaneous opening of the MscL mutant G22C and MscS (5.0 nS). However, when MTSET is attached to MscL mutant G22C, the channel properties change significantly as shown in FIGS. 10B and 10D. First, pressure sensitivity decreases significantly, resulting in spontaneous gating of the channel. Second, when the MscL channel opens, it closes much faster and results in smaller dwell times. It appears that the introduced charge at amino acid position 22 destabilizes both the closed and open state. As a consequence, the channel rapidly switches from the closed to the open state, resulting in this “flickery” appearance even in the absence of membrane tension.

### Example 2B

Examples 1 and 2A showed that specific attachment of MTSET to MscL mutant G22C in spheroplasts results in spontaneous gating of the channel. Patch clamp also showed that the channel opening is exhibiting much smaller dwell times compared to unlabeled channel proteins. Example 2B shows that after MscL purification and membrane reconstitution into an artificial lipid membrane, attachment of MTSET, MTSEA, or MTSES to MscL mutant G22C results in spontaneous gating. Additionally, it is shown that the charge-induced channel opening can result in the release of a membrane impermeable hydrophilic molecule from artificial liposomes containing MscL mutant G22C upon the introduction of a charge by means of an MTS compound.

Materials and Methods

Samples were prepared, and calcein efflux was monitored as described in examples 1 and 2A.

Results and Discussion

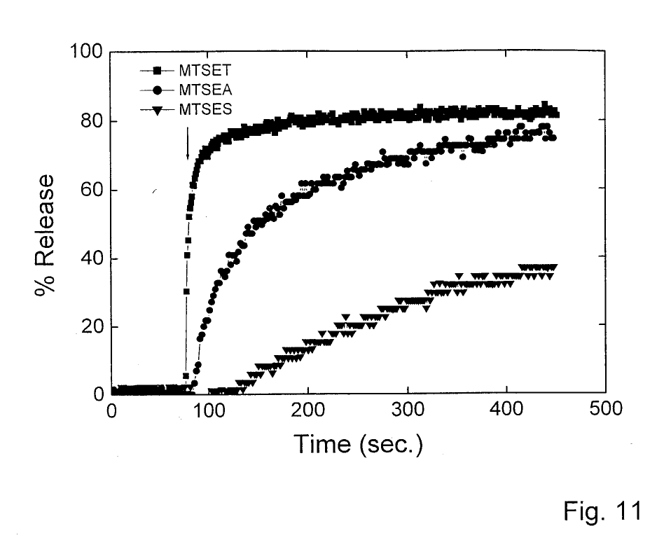

Increase in MscL mediated calcein release upon attachment of MTSET, MTSEA or MTSES to MscL mutant G22C is shown in FIG. 11. MscL mutant G22C reconstituted into DOPC:DOPS (90:10, mol/mol) liposomes showed no calcein release at the time scale of this experiment as indicated by the stable fluorescence in the first 85 sec. of the experiment. At 85 sec., 1 mM MTSET was added to the sample and calcein was rapidly released. In control liposomes, the same lipid composition was used but without MscL and no calcein release was observed (data not shown). This experiment shows that when formulated as described, 80 percent of the encapsulated calcein is effectively released from these liposomes, and half of the MscL mediated efflux occurs within 7 seconds. This experiment was repeated using another positively charged compound MTSEA and a negatively charged compound MTSES. Calcein is released with both these compounds; however, the kinetics of release are significantly slower. The release of calcein upon the addition of these compounds is a composite of the reactivity of the MTS compounds with cysteine at position 22, opening of the MscL channel in response to the introduced charge and the efflux of calcein through the MscL channel. The difference in calcein release can be explained by the difference in hydrophobicity between the MTS compounds, with MTSET being the most hydrophilic and resulting in the fastest cysteine reactivity leading to the fastest calcein release.

These results establish the correlation between the gating properties as determined with patch clamp and the release profile of a hydrophilic molecule from liposomes. It may be concluded that even when a channel exhibits short dwell times, hydrophilic molecules of approximately 600 Da can be effectively and rapidly released under control of charging amino acid at position 22 of the MscL channel.

These results show that liposomes with MscL mutant G22C can be used in a two component system. In this two component system, the first component, liposomes with MscL mutant G22C with encapsulated drug are administered. After these liposomes have accumulated at a target site, a second component is administered. The second component, MTS compound or a similar compound in that attaches specifically to the cysteine at position 22, effectively causes the release of the encapsulated drugs. Using second components of different hydrophobicities allows tailor making of the drug release profiles as shown in FIG. 11.

Example 3

Formulation of liposomes containing MscL channel proteins with an encapsulated substance.

Employing MscL channel proteins for sustained and controlled release of substances is shown in examples 1, 2, 4, 6, and 8. For implementation, it is important to establish a relatively simple procedure to formulate the delivery vehicles. This example shows that mixing synthetic lipids, detergent, MscL channel protein, and the substance that needs to be delivered, followed by detergent removal results in a functional controllable drug delivery vehicle. Additionally, depending on the clinical application, specific lipid compositions of the liposomal drug delivery vehicle may be required. Example 3 shows that the MscL mediated controlled release of a substance can be achieved in liposomes composed of different lipid compositions.

Materials and Methods

MscL mutant G22C was overexpressed and purified as described in example 2. Membrane reconstitution was started by mixing 200 μL of 20 mg/mL of preformed liposomes (DOPC:Cholesterol, 80:20, mol/mol), 500 μL of 0.4 mg/mL purified MscL mutant G22C, 12 mg n-octyl 13-glucoside, and 700 μL of a calcein loading buffer, containing 200 mM calcein, 300 mM sucrose, 25 mM Tris, and 1 mM EDTA, pH 8.0. The membrane reconstitution mixture was incubated for 30 min. at room temperature and 40 mg of detergent-absorbing Bio-Beads SM-2 (Bio-Rad, Inc.) was added and incubated at 4° C. for 30 min. Next, 400 mg Bio-Beads were added and incubated overnight at 4° C. All steps were performed under mild agitation. Samples were prepared for calcein efflux assay as described in example 1.

Results and Discussion

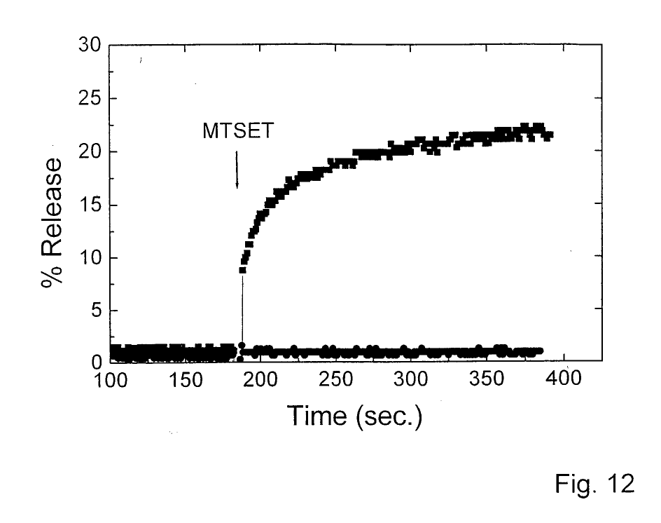

The MscL-mediated release from liposomes was examined by monitoring the increase in fluorescence of the self-quenching fluorescent dye calcein. Upon addition of MTSET to liposomes, as indicated by the arrow of FIG. 12, with and without MscL mutant G22C, only the liposomes with the channel protein exhibited effective release of calcein (FIG. 12). Therefore, mixing all necessary components including the detergent, followed by detergent removal, results in a controllable drug delivery vehicle. Additionally, the MscL mutant G22C can be used for controlled delivery of a substance in DOPC:Cholesterol (80:20, mol/mol) as well as in DOPC:DOPS (90:10, mol/mol) as shown in examples 1 and 2.

### Example 4

pH and light-responsive drug delivery, mediated by chemically modified MscL channel proteins.

It was previously shown that substitution of a residue that resides within the channel pore constriction, Gly-22, with all other 19 amino acids affects channel gating according to the hydrophobicity of the substitution (Yoshimura et al.). One mutant of particular interest for clinical applications was the MscL mutant G22H because it exhibited a significantly higher open probability at pH 6.0 as compared to pH 7.5. This MscL mutant G22H would be an interesting candidate to deliver drugs at target sites with a lowered pH value occurs, such as in solid tumors or at sites of inflammation. When overexpressing this MscL mutant G22H, it was apparent that directly after induction with IPTG, cells would stop growing and the amount of expressed protein was very low and only detectable by western blot analysis (data not shown). Changing growth conditions with respect to medium pH or osmolality did not improve the expression of the MscL mutant G22H.

Examples 1, 2, and 3 showed that MscL mutant G22C can be overexpressed and purified to a high enough yield to be applicable in a drug delivery vehicle. Additionally, this mutant allows the specific attachment of an MTS compound, thus introducing a charge and consequently releasing the substance from the liposomes (FIG. 11 and FIG. 12). A decrease of the pH from 7.5 to 6.0 shifts the equilibrium from the unprotoned to the protonated state of the imidazole side chain of the MscL mutant G22H. This protonation results in the introduction of a charge at amino acid position 22 and affects the opening of the MscL channel. Example 4 shows the chemical synthesis of compounds, reactive specifically with cysteine at amino acid position 22, which introduce chemical groups responsive to pH or light, thus affecting the local hydrophobicity at the pore constriction and the gating of the channel protein.

### Example 4A

This example shows the chemical synthesis of a compound that is reactive specifically with cysteine at amino acid position 22 and contains an imidazole group, effectively mimicking the MscL mutant G22H and circumventing the low production yield of the channel protein.

Materials and Methods

MscL mutant G22C was overexpressed, purified and membrane reconstituted as described in example 2. For labeling of MscL mutant G22C, protein is isolated as described in example 2, but before elution, the column is washed with 10 ml of the wash buffer without imidazole. The label is dissolved to a 1 mg/ml final concentration in the same buffer. The wash buffer in the column is allowed to equilibrate over the column matrix. An equal volume of the buffer containing the label is applied to the column matrix. The top of the column is closed after equilibration with nitrogen gas. The column is incubated at 4° C. for three days and the elution procedure is performed as described in example 2.

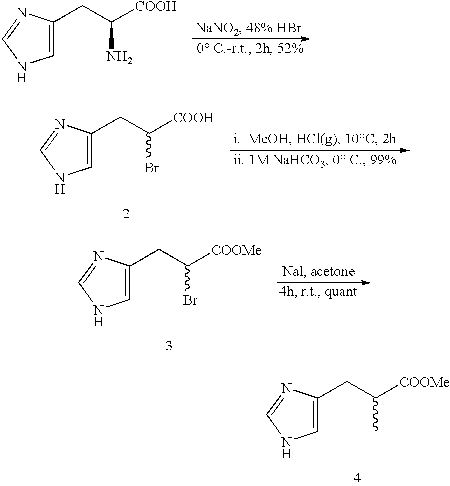

2-bromo-3-(5-imidazolyl)propionic acid monohydrate (mol. 2) (Yankeelov et al. and Maat et al.). For ease of explanation, compounds illustrated herein may also be referred to as (mol. x).

To a vigorously stirred suspension of L-histidine (7.76 g, 50 mmol) in HBr (48%, 110 ml) kept at −5 to 0° C., a solution of NaNO₂ (10.4 g, 150 mmol) in water (20 ml) was dropwise added. After the addition of the NaNO₂, the solution was stirred for 1 hour at 0° C., 1 hour at room temperature and concentrated in vacuo below 50° C. leaving oil with precipitate. This residue was extracted with acetone (4×15 ml), acetone extracts were evaporated in vacuo, and water (20 ml) was added and evaporated in vacuo below 50° C. The residue was dissolved in water (30 ml) and pH was adjusted to 4.6 by aq. ammonia (2M) at 0° C. The solution was evaporated to dryness in vacuo below 50° C. and the solid residue was triturated with ice-cold water (2×30 ml). After drying in vacuo (24 hours, room temperature), the yield of 2-bromo-3-(5-imidazolyl)propionic acid monohydrate (mol. 2) was 6.20 g, 52%.

Methyl 2-bromo-3-(5-imidazolyl)propanoate (mol. 3) (Maat et al.).

Through a stirred solution of 2-bromo-3-(5-imidazolyl)propionic acid monohydrate (mol. 2) (5 g, 21.1 mmol) in methanol (75 ml) kept at 11° C. was bubbled dry HCl for 2 hours. The solution was evaporated in vacuo below 50° C., the resulting oil was dissolved in aq. NaHCO₃ (1M, 75 ml) and extracted with chloroform (3×50 ml). The extracts were dried over Na₂SO₄, filtered and evaporated in vacuo below 50° C. to yield methyl 2-bromo-3-(5-imidazolyl)propanoate (mol 3) as a slightly yellow oil (4.87 g, 99%).

Methyl 2-iodo-3-(5-imidazolyl)propanoate (mol. 4).

A solution of NaI (3 g, 20 mmol) in acetone (10 ml) was added to a solution of methyl 2-bromo-3-(5-imidazolyl)propanoate (mol. 3) (2.33 g, 10 mmol) in acetone (10 ml). Reaction mixture was stirred and protected from light. After 4 h, reaction mixture was evaporated in vacuo to dryness, and the residue was dissolved in water (15 ml) and extracted with ethyl acetate (3×15 ml). Combined extracts were washed with aq. Na₂S₂O₃ (1M, 5 ml), dried over Na₂SO₄, and evaporated in vacuo at room temperature to give methyl 2-iodo-3-(5-imidazolyl)propanoate (mol. 4) as a slightly yellow oil (2.80 g, 100%).

Results and Discussion

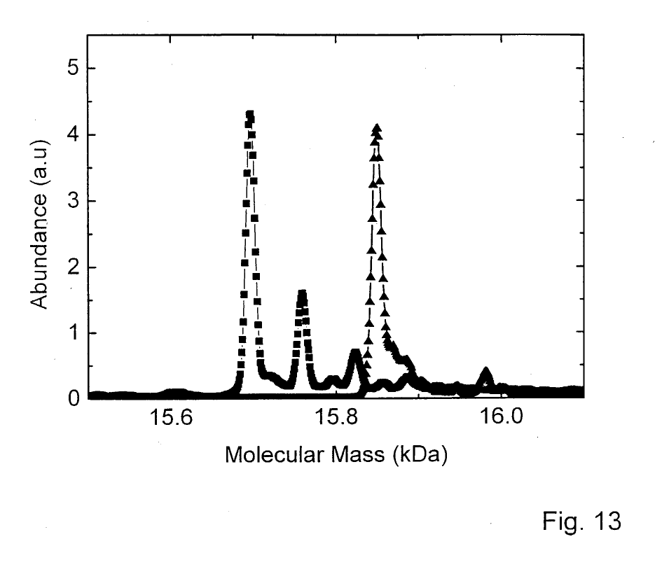

MscL mutant G22C was labeled with 2-bromo-3-(5-imidazolyl)propionic acid monohydrate (BI) or methyl 2-iodo-3-(5-imidazolyl)propanoate (IMI) for three days and products were analyzed using ESI-MS. The product of the incubation with BI showed a mass identical to the calculated mass of unlabeled MscL mutant G22C (data not shown). The product of the incubation with IMI showed a mass calculated for the MscL mutant G22C with an expected additional mass of 153 Da, indicative of proper labeling (FIG. 13). Additionally, no unlabeled protein was observed after labeling with IMI under the described conditions and no doubly labeled subunits were observed, indicating that labeling conditions are optimal for IMI. The absence of attachment with BI, which is less hydrophobic than IMI, is consistent with the observations in example 2, FIG. 11, where cysteine reactivity relates to the hydrophobicity of the label.

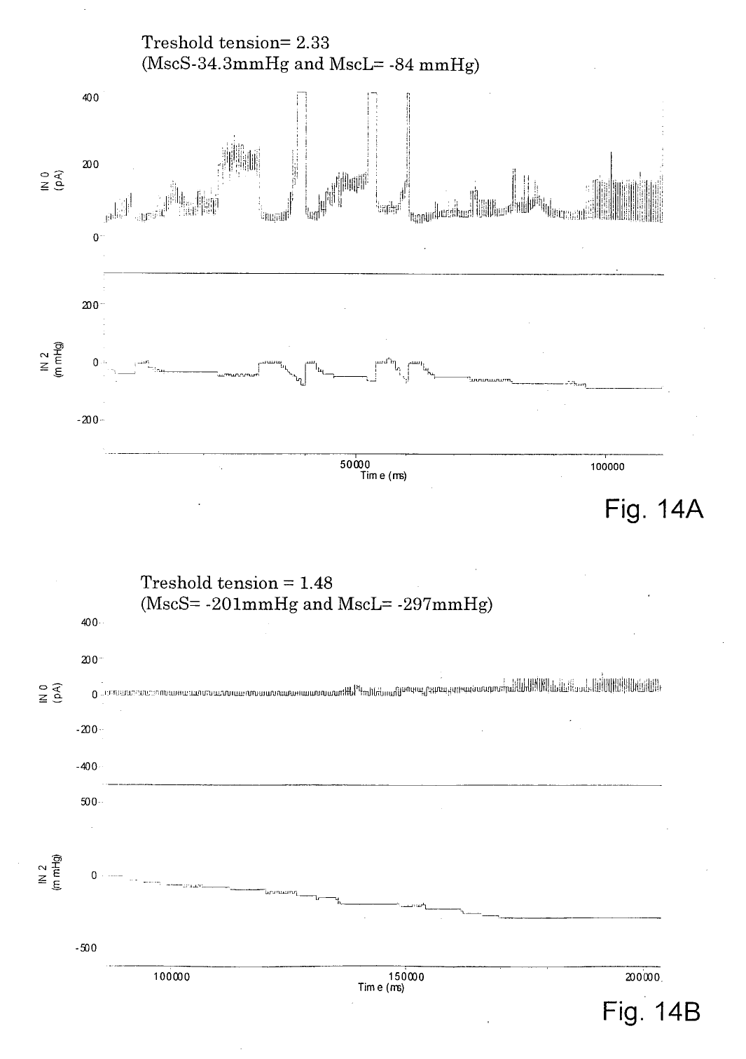

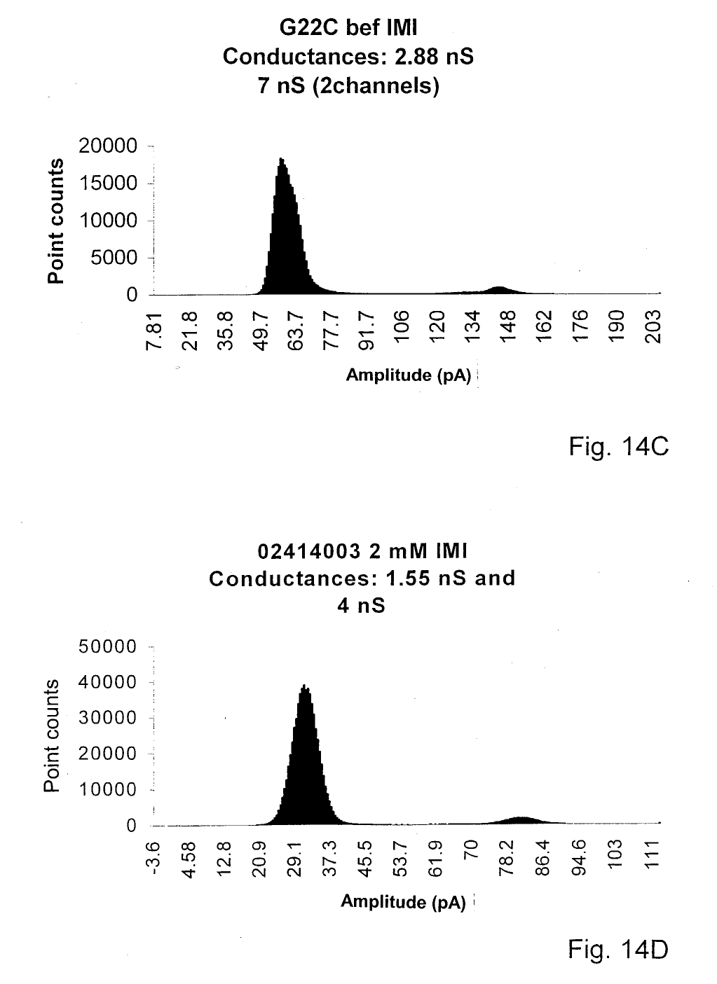

IMI labeled MscL mutant G22C in spheroplast were analyzed using patch clamp to characterize the channel properties as shown in FIG. 14.

Patch clamp experiments on spheroplast allows quantitation of the tension sensitivity because of the presence of an internal control, which is the mechanosensitive channel of small conductance (MscS). By labeling the MscL mutant G22C with IMI, the ratio of tension sensitivity of MscL over MscS significantly decreases from 2.33 to 1.48. This result indicates that the IMI labeling effectively makes the channel protein open at a lower membrane tension at pH 6. Additionally, calcein efflux assays showed that the channel still has a tendency to stay open at pH 7.0 and 8.0 (data not shown.) For most clinical applications, it is important that the channel remains closed at pH values of 7.4. Therefore, another compound with a lower pKa was designed and synthesized, see example 4B.

### Example 4B

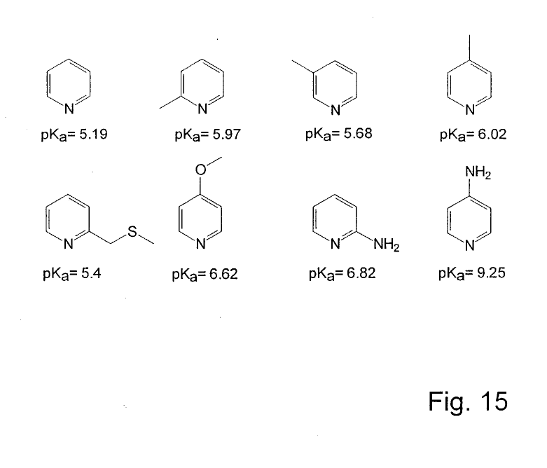

The pKa of the IMI group attached to the MscL mutant G22C controls the gating of the MscL channel and therefore also controls the drug release in response to the pH. To manipulate this pH sensitivity of the drug delivery vehicle, several other pH sensitive compounds were designed (FIG. 15). The pKa's of these compounds are very diverse and allows for fine-tuning of the drug release profile to the specific clinical application.

Materials and Methods

MscL mutant G22C was overexpressed, purified, labeled, and membrane reconstituted as described in examples 2 and 4A. Synthesis of one of the substituents described in FIG. 15 is described below.

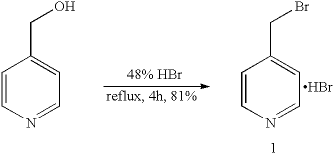

4-(bromomethyl)pyridine hydrobromide (mol. 1) (Bixler et al.).

4-pyridinylmethanol (2 g, 18.3 mmol) was dissolved in aq. HBr (48%, 20 ml), the solution was refluxed for 4 hours and concentrated in vacuo. The semisolid material was triturated with absolute ethanol (10 ml), cooled to 0° C., filtered and washed with another portion of ice cooled absolute ethanol (10 ml). After drying in vacuo, the yield of 4-(bromomethyl)pyridine hydrobromide (mol. 1) was 3.67, 81%.

Results and Discussion

Labeling was optimized for this pyridine compound, 4-(bromomethyl)pyridine hydrobromide (BP), to MscL mutant G22C and ESI-MS showed that all channel proteins were labeled (data not shown). Patch clamp was used to characterize the effect of this label on the channel gating properties.

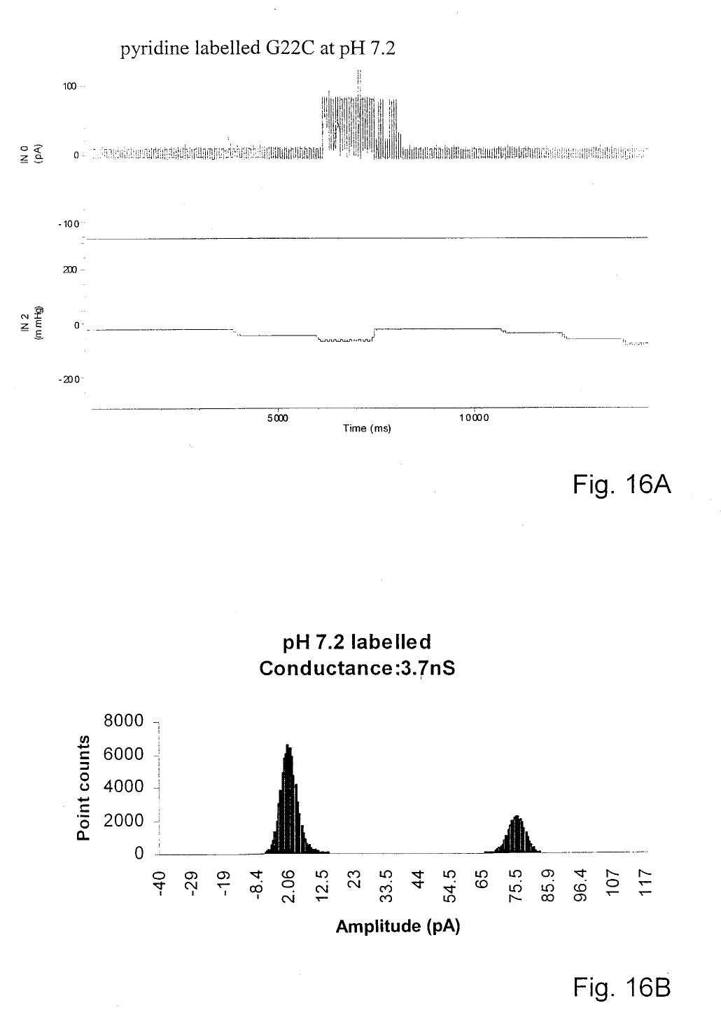

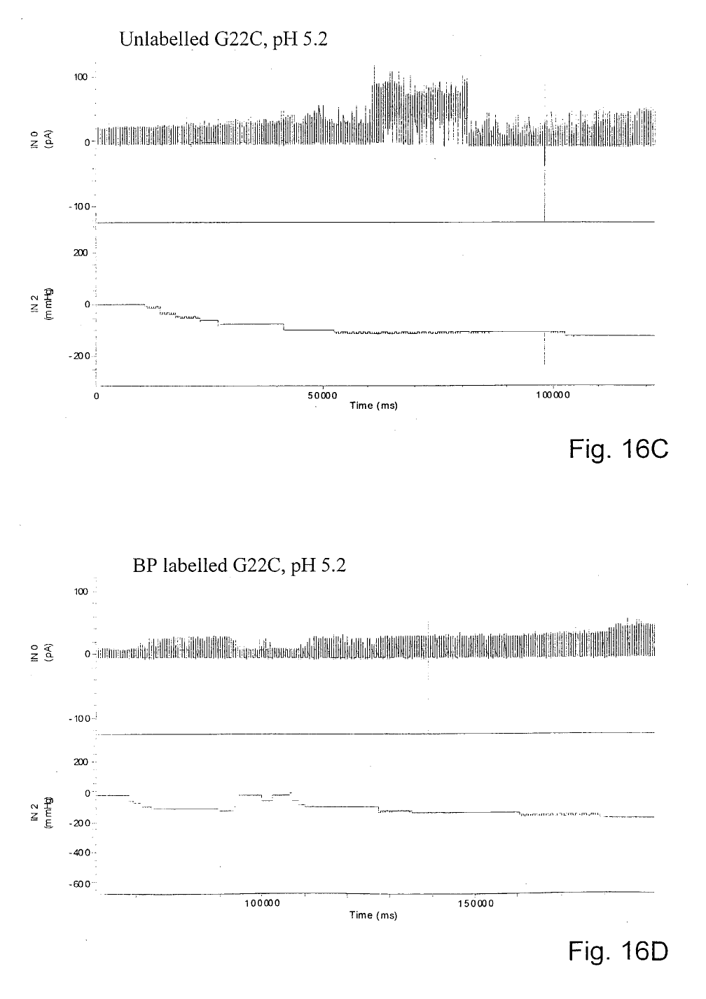

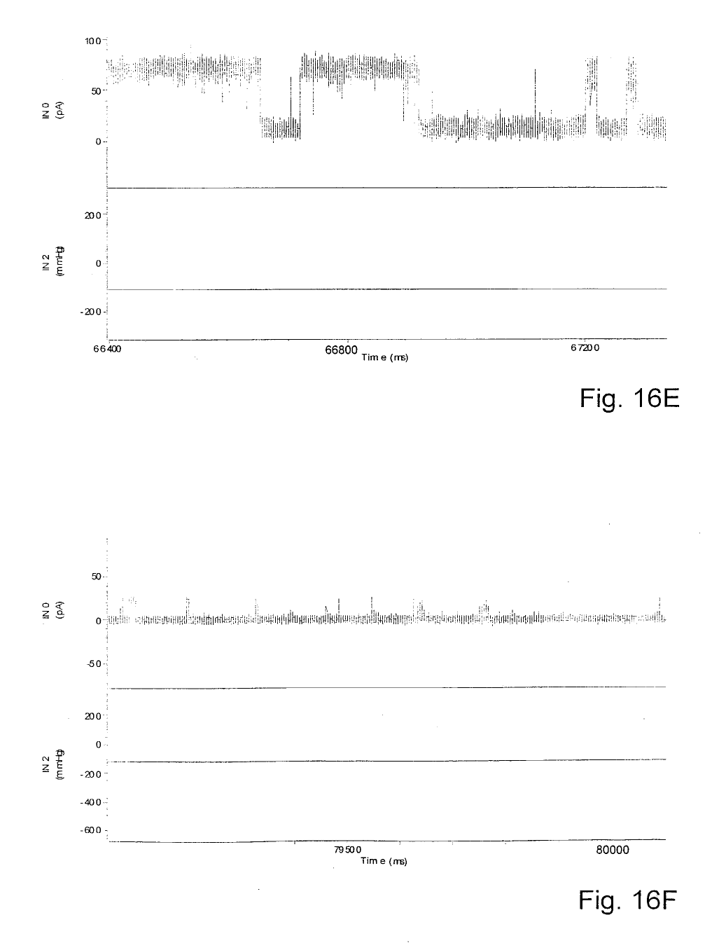

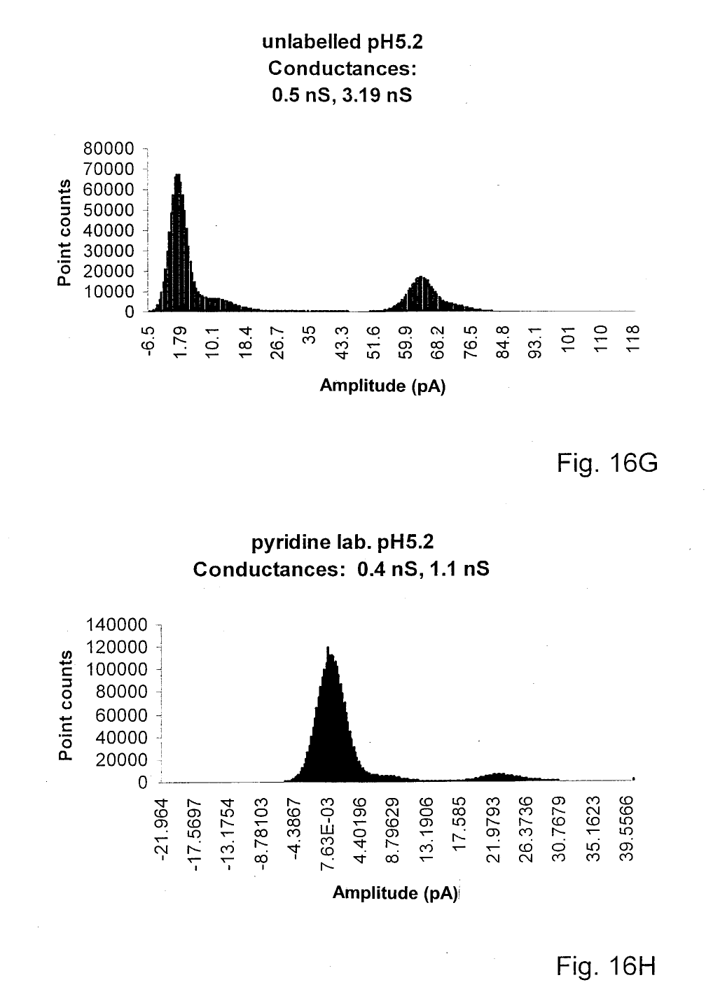

Upon labeling with BP, the MscL mutant G22C channel shows a pH dependent change (FIG. 16). At pH 7.2, the BP labeled channel behaves as an unlabeled channel by exhibiting the same type of conductance preference and dwell times. However, when the channel is analyzed at pH 5.2, it prefers not to open completely but only to subconducting states and the dwell times get shorter. Comparing the behavior of BP labeled protein at pH 5.2 to both unlabeled protein at pH 5.2 and labeled proteins at pH 7.2 indicates that the channel opens normally at high pH values, but at lower pH values the channel starts to open more readily with shorter dwell times. The behavior of the BP labeled channel at low pH is very similar to the MTSET labeled channel (FIG. 11), whereas at higher pH values, the BP labeled channel behaves as unlabeled channel protein. Therefore, it can be concluded that this BP labeled MscL mutant G22C will release drugs comparable to the MTSET induced release of calcein or insulin (examples 2 and 8, respectively) at low pH, whereas at higher pH values, the channel is tightly closed, ensuring little or no release of these substances.

### Example 4C

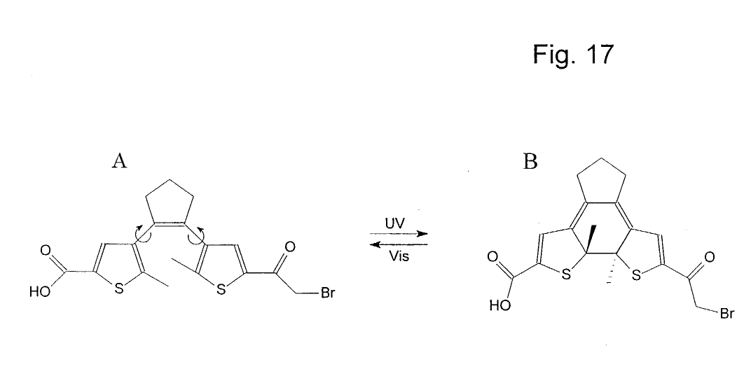

Instead of using the compounds described in examples 2, 3, 4A, and 4B, photoreactive compounds can be designed to react with MscL mutant G22C and respond to the absorption of light by changing the local charge or hydrophobicity. An example of such a photoreactive molecule is 4-{2-[5-(2-Bromo-acetyl)-2-methyl-thiophen-3-yl]-cyclopent-1-enyl}-5-methyl-thiophene-2-carboxylic acid (DTCP1), which was designed and synthesized to reversibly switch conformation after light absorption of specific wavelengths (FIG. 17).

Materials and Methods

MscL mutant G22C was overexpressed, purified, labeled, and membrane reconstituted as described in examples 2 and 4A.

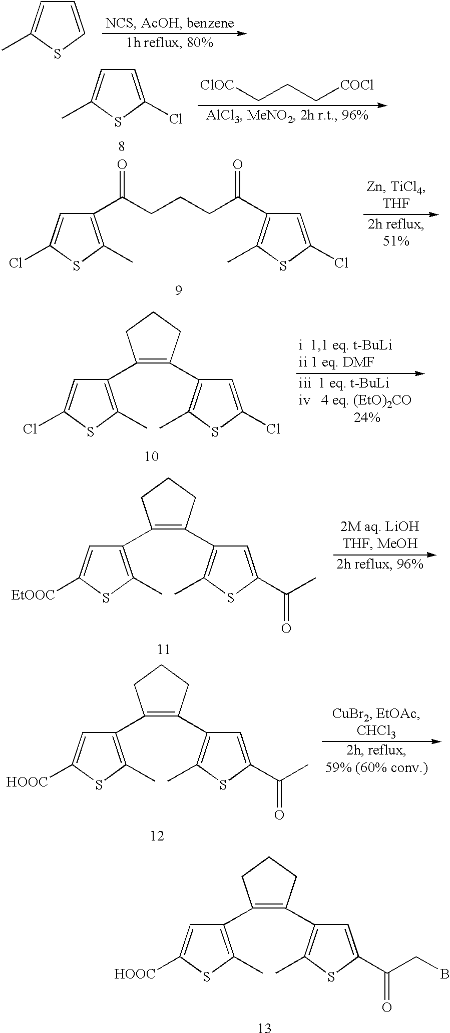

A suspension of N-chlorosuccinimide (75.9 g, 0.568 mol) and 2-methylthiophene (50 ml, 50.7 g, 0.516 mol) in a mixture of benzene (200 ml) and acetic acid (200 ml) was stirred for 30 minutes at room temperature and then 1 hour at reflux temperature. The cooled mixture was poured into aq. NaOH (3 M, 150 ml), the organic phase was washed with NaOH (3M, 3×150 ml), dried over Na₂SO₄ and evaporated in vacuo. A slightly yellow liquid product was purified by vacuum distillation (19 mm, 55° C.) to produce a colorless liquid of 2-chloro-5-methylthiophene (mol. 8) (55 g, 80.3%).

1,5-bis(5′-chloro-2′-methylthien-3′-yl)pentadione (mol. 9) (Lucas et al.).

To a solution of 2-chloro-5-methylthiophene (mol. 8) (32.3 ml, 39.8 g, 0.3 mol) and glutaryl dichloride (19.2 ml, 25.4 g, 0.15 mol) in nitromethane (300 ml), AlCl₃ (48 g, 0.36 mol) was added at 0° C. under vigorous stirring in several portions. After 2 hours of stirring at room temperature, ice-cold water (150 ml) was added and extracted with diethyl ether (3×150 ml). Combined ether extracts were washed with water (100 ml), dried over Na₂SO₄ and evaporated in vacuo to yield a brown tar (52 g, 96%). This crude 1,5-bis(5′-chloro-2′-methylthien-3′-yl)pentadione (mol. 9) was used further without purification.

1,2-bis(5′-chloro-2′-methylthien-3′-yl)cyclopentene (mol. 10) (Lucas et al.).

To a Zn dust (10 g, 0.153 mol) suspension in dry THF (200 ml) in a three-neck flask under nitrogen was slowly added TiCl₄ (24.8 ml, 42.9 g, 0.226 mol) through a glass syringe The resulting mixture was refluxed for 45 minutes. The flask was cooled in an ice bath and crude 1,5-bis(5′-chloro-2′-methylthien-3′-yl)pentadione (mol. 9) (27.4 g, 75.9 mmol) was added. After refluxing for 2 hours, the reaction was quenched with aq. K₂CO₃ (10%, 200 ml) and extracted with diethyl ether (4×80 ml). Combined organic extracts were washed with water (100 ml), dried over Na₂SO₄ and evaporated in vacuo. After column chromatography on silica-gel (petroleum ether 40-60) 1,2-bis(5′-chloro-2′-methylthien-3′-yl)cyclopentene (mol. 10) was obtained as a white solid (12.6 g, 50%).

ethyl 4-[2-(5-acetyl-2-methyl-3-thienyl)-1-cyclopenten-1-yl]-5-methyl-2-thiophenecarboxylate (mol. 11).

To a mixture of 1,2-bis(5′-chloro-2′-methylthien-3′-yl)cyclopentene (mol. 10) (700 mg, 2.13 mmol) in diethyl ether (50 ml), t-BuLi (1.5M in pentane, 1.7 ml) was added at 0° C. After 10 min., the cooling bath was removed and the reaction mixture was stirred for 50 min. at room temperature. N,N-dimethylacetamide (0.2 ml, 195 mg, 2.23 mmol) was added at 0° C., stirred at 0° C. for 10 min. and stirred for 50 min. at room temperature. Again, t-BuLi (1.5 M in pentane, 1.4 ml) was added at 0° C. and stirred for 10 min. and stirred for 50 min. at room temperature. Diethyl carbonate (1 ml, 957 mg, 8.25 mmol) was added at 0° C., stirred at 0° C. for 10 min. and stirred for 50 min. at room temperature. The reaction was then quenched with aq. HCl (1 M, 20 ml), the organic layer was separated and the water layer was extracted with diethyl ether (3×20 ml). Combined organic layers were washed with saturated aq. NaHCO₃ (10 ml), dried over Na₂SO₄ and evaporated in vacuo. After column chromatography on silica-gel (hexane:ethyl acetate/9:1), ethyl 4-[2-(5-acetyl-2-methyl-3-thienyl)-1-cyclopenten-1-yl]-5-methyl-2-thiophenecarboxylate (mol. 11) was obtained (192 mg, 24%).

4-[2-(5-acetyl-2-methyl-3-thienyl)-1-cyclopenten-1-yl]-5-methyl-2-thiophenecarboxylic acid (mol. 12).

To a solution of ethyl 4-[2-(5-acetyl-2-methyl-3-thienyl)-1-cyclopenten-1-yl]-5-methyl-2-thiophenecarboxylate (mol. 11) (114 mg, 0.315 mmol) in a mixture of THF (3 ml) and methanol (1 ml), aq. LiOH (2 M, 0.6 ml) was added and the mixture was refluxed for 24 hours. Aq. HCl (1 M, 10 ml) was added and extracted with ethyl acetate (3×10 ml). Organic extracts were washed with water (5 ml), dried over Na2SO₄ and evaporated in vacuo. After column chromatography on silica-gel (hexane:ethyl acetate/1:1, then CH₂Cl₂: methanol/9:1), 4-[2-(5-acetyl-2-methyl-3-thienyl)-1-cyclopenten-1-yl]-5-methyl-2-thiophenecarboxylic acid (mol. 12) was obtained (101 mg, 92%).

4-{2-[5-(2-bromoacetyl)-2-methyl-3-thienyl]-1-cyclopenten-1-yl}-5-methyl-2-thiophenecarboxylic acid (mol. 13).

To a boiling suspension of finely grounded CuBr₂ (130 mg, 0.581 mmol) in ethyl acetate (2 ml), a solution of 4-[2-(5-acetyl-2-methyl-3-thienyl)-1-cyclopenten-1-yl]-5-methyl-2-thiophenecarboxylic acid (mol. 12) (101 mg, 0.292 mmol) was added with vigorous stirring in chloroform (2 ml). After 2 hours of reflux, the mixture was filtered and evaporated. After column chromatography on silica-gel (CH₂Cl₂:methanol/99:1), 4-{2-[5-(2-bromoacetyl)-2-methyl-3-thienyl]-1-cyclopenten-1-yl}-5-methyl-2-thiophenecarboxylic acid (mol. 13) was isolated (73 mg, 59%) together with starting material (40 mg, 40%).

Nuclear Magnetic Resonance and spectroscopic analysis (data not shown) indicated DTCP 1 was chemically and functionally correct as shown in FIG. 17.

Results and Discussion

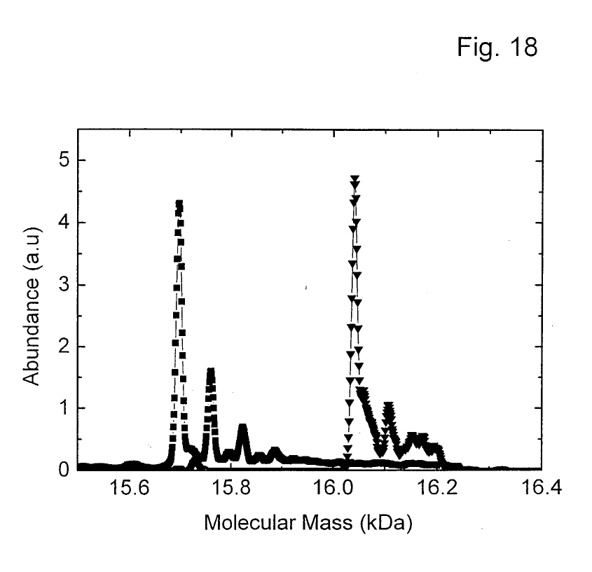

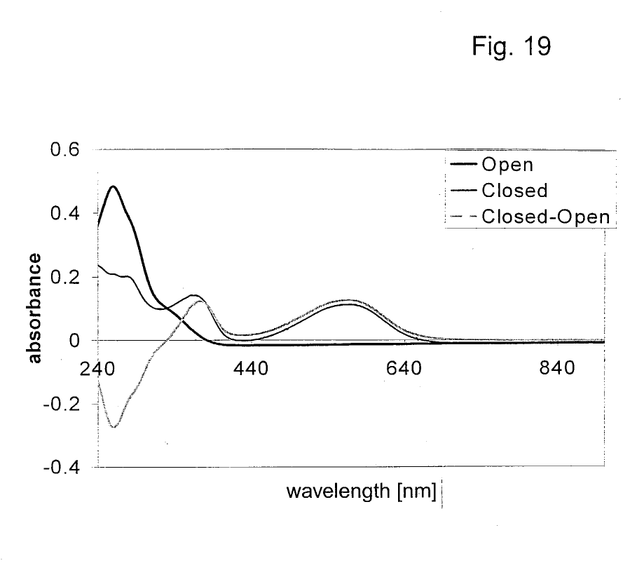

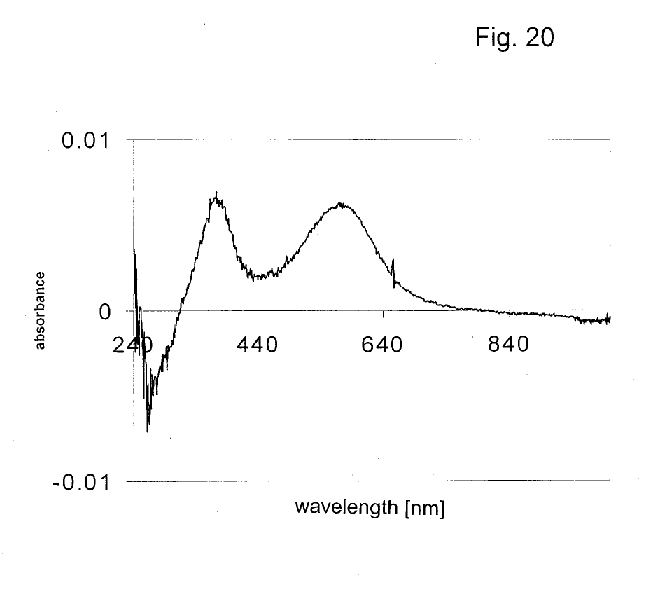

DTCP1 was designed to specifically react with the free sulfhydryl group of a single cysteine at position 22 of MscL (G22C-MscL). Position 22 in the MscL channel was chosen for its involvement in the gating mechanism of the channel. A conjugation protocol was developed and the products were analyzed employing electrospray ionization mass spectrometry (ESI-MS) and absorption spectroscopy. ESI-MS indicated that the mass of all MscL subunits increased 344 Da. A mass increase is expected for a conjugation of DTCPI to a sulfhydryl group of MscL as shown in FIG. 18. The two photo-isomers of DTCP1 exhibit different absorption spectra in the UV region as shown in FIG. 19. This difference was used to monitor the switching of DTCP1 after conjugation to MscL and reconstitution of the detergent-solubilized G22C-MscL-DTCP1 conjugate into DOPC:DOPS (90:10, mol/mol) containing lipid bilayer as shown in FIG. 20 (due to light scattering by liposomes, only substracted spectra before and after irradiation can be shown).

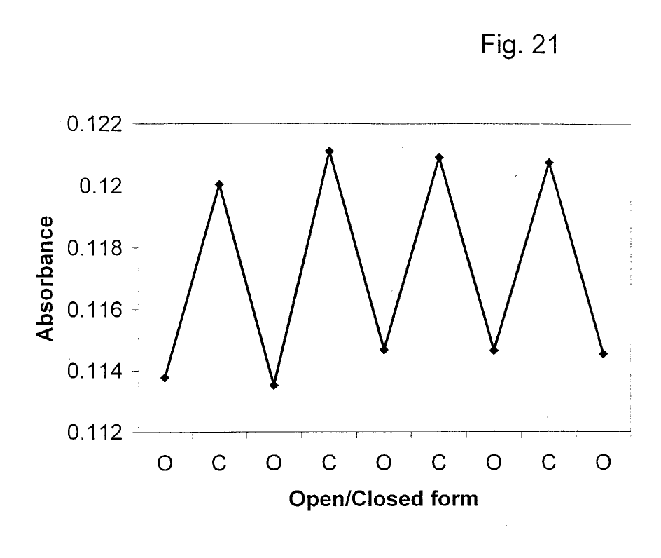

As can be seen from FIG. 20, conjugation and reconstitution into lipid bilayer has no effect on switching of DTCP 1. To prove reversibility and reproducibility of switching, this system was repeatedly irradiated with 313 nm UV light to achieve closed form and with light of a wavelength longer than 460 nm to return back to open form while monitoring at 535 nm (absorption maximum of closed form) as shown in FIG. 21.

The data shows that an organic molecule (DTCP1) has been synthesized and that this molecule can be conjugated to a specific site in the MscL channel, known to alter the gating properties of the channel, while maintaining the desired photochemical properties.

### Example 4D

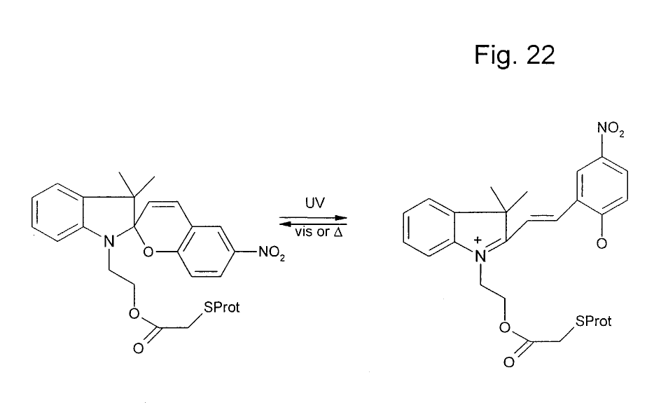

The DTCP1 molecule (example 4C) contains a free carboxylic group in order to modify hydrophobicity of the pore of MscL. To enhance the hydrophilic properties of the synthesized molecule, spiropyran derivative SP1 was prepared (FIG. 22) which changes into highly charged merocyanine form after UV irradiation.

Materials and Methods

MscL mutant G22C was overexpressed, purified, labeled, and membrane reconstituted as described in examples 2 and 4A, except labeling on the column was 30 min. instead of 3 days.

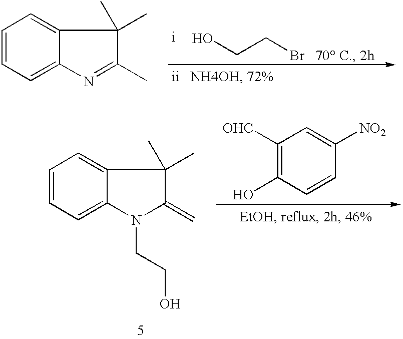

2-(3,3-dimethyl-2-methylene-2,3-dihydro-1H-indol-1-yl)-1-ethanol (mol. 5) (Sakuragi et al.).

The mixture of 2,3,3-trim&thyl-3H-indole (5 g, 31.4 mmol) and 2-bromoethanol (2.22 ml, 3.92 g, 31.4 mmol) was heated with stirring at 70° C. for 2 hours, cooled to room temperature and washed with aq. ammonia (25%, 25 ml). Separated yellow oil was extracted with diethyl ether, dried over Na₂SO₄ and evaporated to give 2-(3,3-dimethyl-2-methylene-2,3-dihydro-1H-indol-1-yl)-1-ethanol (mol. 5) as an oil (4.57 g, 72%).

2-(3,3-dimethyl-6-nitrospiro[2H-1-benzopyran-2,2′indoline])-1-ethanol (mol. 6) (Sakuragi et al.).

The solution of 2-(3,3-dimethyl-2-methylene-2,3-dihydro-1H-indol-1-yl)-1-ethanol (mol. 5) (2 g, 9.8 mmol) and 2-hydroxy-5-nitrobenzaldehyde (1.64 g, 9.8 mmol) in ethanol (50 ml) was refluxed for 2 hours. After filtration, the product was recrystalized from ethanol. Yield of pure 2-(3,3-dimethyl-6-nitrospiro [2H-1-benzopyran-2,2′indoline])-1-ethanol (mol. 6) was (1.6 g, 46%).

2-(3,3-dimethyl-6-nitrospiro[2H-1-benzopyran-2,2′indoline])ethyl 2-bromoacetate (mol. 7).

The solution of 2-(3,3-dimethyl-6-nitrospiro[2H-1-benzopyran-2,2′indoline])-1-ethanol (mol. 6) (1 g, 2.84 mmol), bromoacetyl bromide (0.37 ml, 0.86 g, 4.26 mmol) and pyridine (0.35 ml, 0.34 g, 4.26 mmol) in toluene (10 ml) was stirred at room temperature for 16 hours. Water (10 ml) was added and extracted with diethyl ether (3×10 ml). Organic extracts were dried over Na₂SO₄, filtered and evaporated in vacuo. After chromatography on silica-gel (hexane:ethyl acetate/5:1), an oily product (mol. 7) (0.65 g, 48%) was obtained.

Results and Discussion

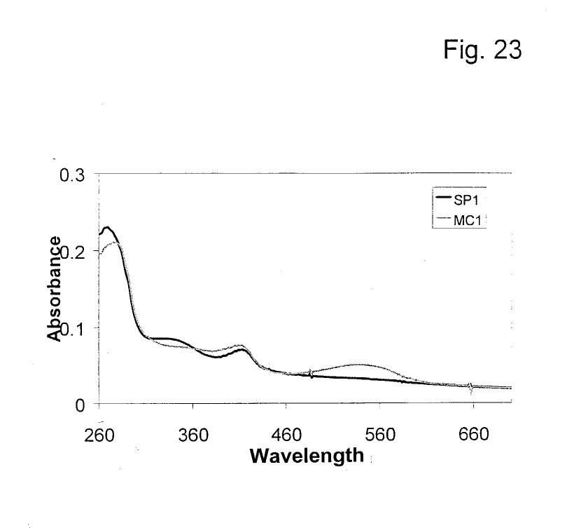

SP1 reacts specifically with the free sulfhydryl group of cysteine at position 22 of MscL, allowing the channel protein to be specifically modified. FIG. 23 shows the UV change of the SP1 conjugated to MscL after irradiation with 313 nm UV light. The new peak at 550 nm belongs to the merocyanine form of the molecule.

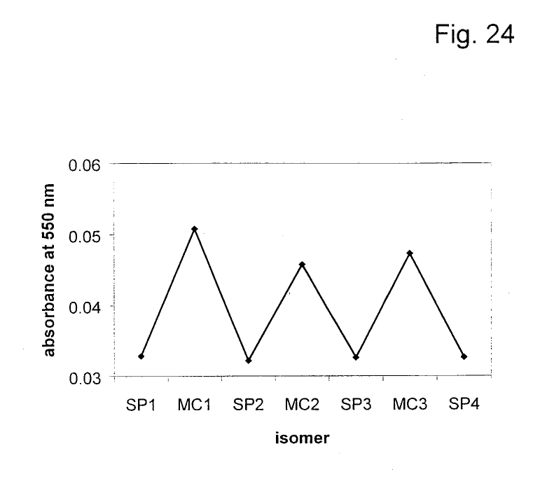

To show reversibility and reproducibility of switching, SP1 conjugated to protein was repeatedly irradiated at 313 nm UV light to achieve merocyanine form and with light with a wavelength longer than 460 nm to return back to spiropyran form while monitoring at 550 nm (absorption maximum of closed form) as shown in FIG. 24.

Materials and Methods

Starting materials were commercially available (Aldrich, Acros Chimica, Fluka) and were used without further purification. Diethyl ether and THF were distilled from Na. For column chromatography, Aldrich silica gel Merck grade 9385 (230-400 mesh) was used.

Compounds (mol. 10-13) are light sensitive and were handled in dark, resp. using brown glassware.

All compounds were characterized using ¹H NMR (Varian VXR-300 at 300 MHz, or Varian Gemini-200 at 200 MHz), ¹³C NMR (Varian VXR-300 at 75.4 MHz, or Varian Gemini-200 at 50.3 MHz), and mass analysis (MS-Jeol mass spectrometer).

### Example 5

Upon exposure to light, a photoreactive lipid alters its chain conformation, which induces a changed lateral pressure in the membrane to control the gating of the MscL channel.

The basic components of this drug delivery vehicle are a lipid membrane and the MscL channel protein. Controlled release of a drug from these vehicles can either be achieved by directly effecting the gating mechanism of the channel protein or indirectly by effecting the physical properties of the lipid bilayer, which subsequently controls the gating of the channel. This example shows that the synthesis of photoreactive lipids, when incorporated in liposomes, can affect the lateral pressure in these membranes and control the gating of the MscL channel protein.

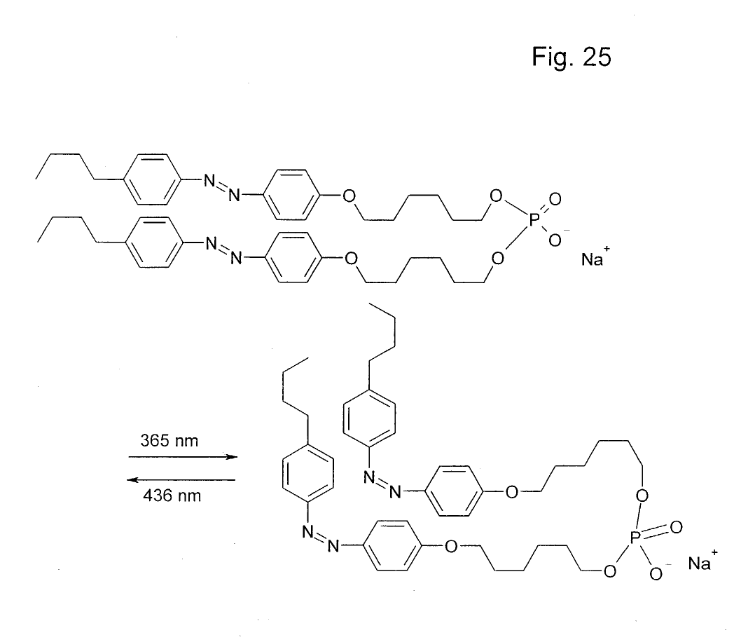

Photoreactive lipids were designed and synthesized to reversibly switch conformation upon radiation with light of an appropriate wavelength (FIG. 25).

Three different lipids with an azobenzene unit have been synthesized (J. M. Kuiper and J. B. F. N. Engberts, to be published).

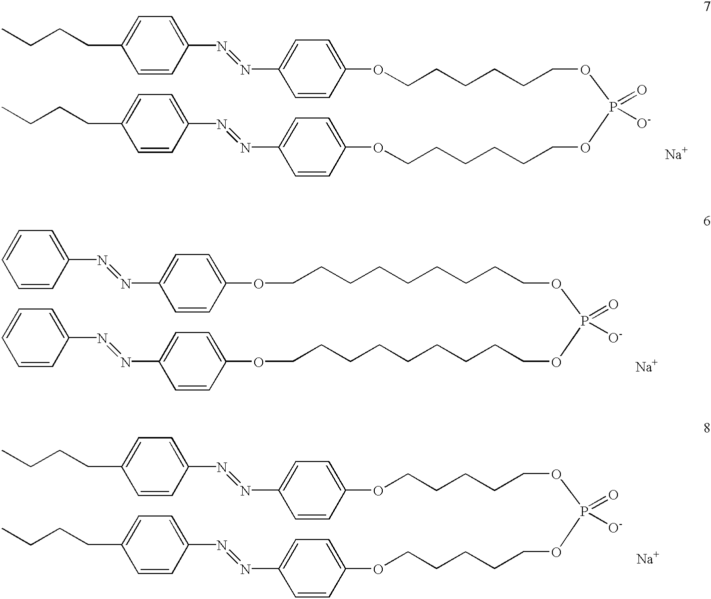

The synthesis of lipid (mol. 6) is described in the experimental section. The synthesis of lipids (mol. 7) and (mol. 8) is similar.

Materials and Methods

Synthesis of (mol. 1). To 4.0 g (20.16 mmol) of 4-phenylazophenol in 150 ml of acetone, 4.52 g (20.16 mmol) 9-bromonona-1-ol, 5.56 g (40.32 mmol) of K₂CO₃ and a catalytic amount of KI was added. The mixture was refluxed for 5 days. The acetone was removed by evaporation under reduced pressure. Dichloromethane (500 ml) was added and the organic layer was washed three times with a fresh layer of water. The organic layer was dried over NaSO₄, filtrated and evaporated under reduced pressure. The resulting yellow solid material was purified by crystallization from ethyl acetate (120 ml). Yellow crystals were obtained in 75% yield and the product was characterized by ¹H and ¹³C NMR.

Synthesis of (mol. 2). To 1.5 g (4.41 mmol) of (mol. 1) in 15 ml of dry dichloromethane under a nitrogen atmosphere, 238 μl (2.94 mmol) of pyridine and 128 μl (1.47 mmol) of PCl₃ was slowly added. The reaction was monitored by TLC (silica, ether) and additional portions of pyridine and PCl₃ (ratio 2:1) were added when the alcohol was still present. After the reaction was completed, the dichloromethane was washed twice with a saturated aqueous solution of NaCl. In the case of very difficult separations, the addition of acid sometimes brought some relief. The organic layer was dried over NaSO₄, filtered and evaporated under reduced pressure. The resulting material was stirred overnight in hexane and the crystals were removed by filtration. These crystals were further purified by crystallization from ethanol. The hot solution of product in ethanol was filtrated. The crystallization took place at room temperature. The crystallization was repeated and pure yellow crystals were obtained in a 53% yield. The product was characterized by ¹H, ³¹P and ¹³C NMR.

Synthesis of (mol. 5). To 0.305 mg (0.42 mmol) of (mol. 2) in 15 ml of tetrachloromethane, 234 μl (1.68 mmol) of triethylamine and 8 μl (10.1 eq) of diisopropylethylamine were added. The mixture was stirred at room temperature for 19 days, even though the reaction time was much shorter (2-3 days), wherein the conversion can be followed by ³¹P NMR. The volatile compounds were removed by evaporation under reduced pressure. To the resulting material (mol. 3), 0.6 ml acetic acid and 3 ml triethylamine were added. After two days of stirring at room temperature, the reaction was completed. Again the volatile compounds were removed by evaporation under reduced pressure. The obtained crude product (mol. 4) was hydrolyzed by stirring in acidic water (pH=4-5, 100 ml) for 30 min. The resulting mixture was subjected to water/dichloroethane (100 ml) extraction. The organic layer was washed twice with a saturated aqueous solution of NaCl. With difficult separations, the addition of some acid was advantageous. The organic layer was dried over NaSO₄, filtrated and evaporated under reduced pressure. The solid material was further purified by crystallization from ethanol. The hot solution of product in ethanol was filtrated. The solution was put in a refrigerator overnight. The crystals were washed with cold ethanol. Yellow crystals were obtained in a 66% yield and the product (mol. 5) was characterized by ¹H, ³¹P and ¹³C NMR.

Synthesis of (mol. 6). To 0.1773 g (0.245 mmol) of (mol. 5), 1.80 g of a sodium ethoxide solution in ethanol (0.136 mmol/g) was added. Extra dry ethanol was added and the solution was slowly warmed up until the solution became clear. After cooling, crystals were formed and the solution was put in the refrigerator. The crystals were washed with cold ethanol. Yellow crystals were obtained in an 89% yield and the product (mol. 6) was characterized by ¹H, ³¹P and ¹³C NMR.

Preparation of the vesicles.

DSP/lipid (mol. 7) (95:5, mol/mol): The appropriate amounts of the lipids were solubilized in methanol. A thin film was created by evaporating the methanol under reduced pressure. Subsequently, the film was kept under a high vacuum for at least one hour. Water was added and the mixture was firmly stirred for one hour at 85° C. At the end, tip sonication was applied (3 times for 30 s) and a clear solution was obtained.

DOPC/lipid (mol. 6) (95:5, mol/mol): The appropriate amounts of the lipids were solubilized in methanol. A thin film was created by evaporating the methanol under reduced pressure. Subsequently, the film was kept under a high vacuum for at least one hour.

Water was added and the mixture was firmly stirred. The mixture was kept at 95° C. for 15 minutes and the mixture was sonicated with a sonicator for a few minutes. A clear solution was obtained.

Results and Discussion

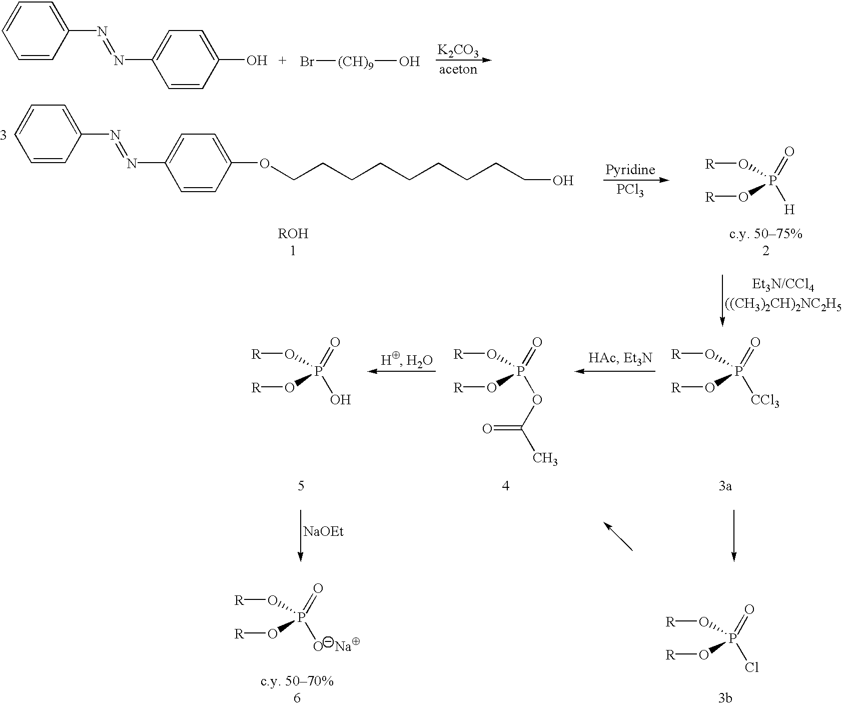

Scheme 2, Synthesis phosphates, see above.

Synthesis: A new synthetic route was used to synthesize the sodium phosphates (Scheme 2). Particularly the combination of the 3^{rd}, 4^{th} and 5^{th} step is new. These steps are very mild steps and can easily be followed by ³¹P NMR. The 3^{rd}, 4^{th} and 5^{th} steps take place with a complete conversion. First, with the use of PCl₃ and pyridine, the phosphonate can be synthesized. With the use of a base and tetrachloromethane, compound (mol. 3a) is obtained. This is called the Atherton Openshaw Todd reaction. (mol. 3a) can react further to (mol. 3b).

After addition of acetic acid and base, a nucleophilic attack of acetic acid takes place at the phosphorus atom. After acid-catalyzed hydrolysis, the phosphate acid is obtained which can be converted into the sodium salt with the use of sodium ethoxide.

Vesicle formation.

It was found that lipids (mol. 6-8) are not vesicle forming. This was confirmed by EM (electron microscopy, data not shown). The lipids were mixed with vesicle-forming lipids (e.g., DOPC, DOP (sodium dioleyl phosphate) and DSP (sodium distearyl phosphate)). With a ratio of 95:5 for vesicle-forming lipids and azobenzene-containing lipids, stable vesicle solutions could be prepared. All mixtures were examined by EM.

UV/Vis spectroscopy and irradiation experiments.

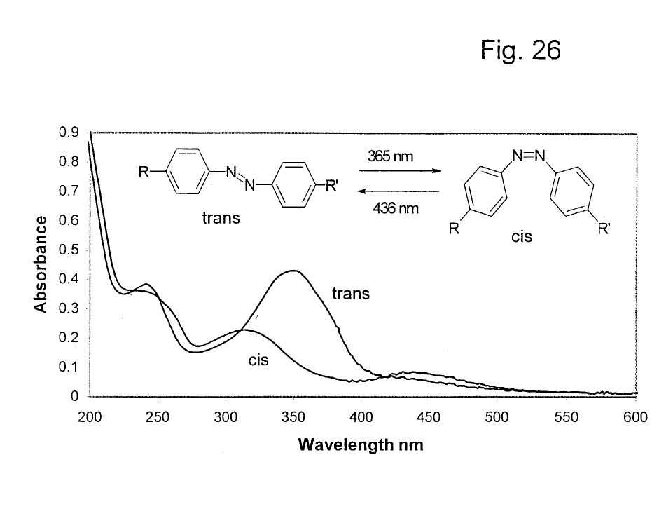

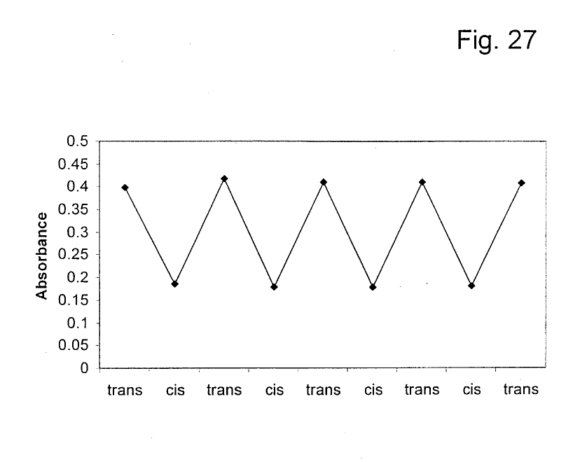

In FIG. 26, the UV/V is absorption spectra of a mixture of DSP and (mol. 7) are shown. The trans isomer was switched into the cis isomer upon irradiation with light of 365 nm. Also, the back isomerization went smoothly. For a mixture of 95% DOPC and 5% lipid (mol. 6), the irradiation cycle was repeated several times (FIG. 27).

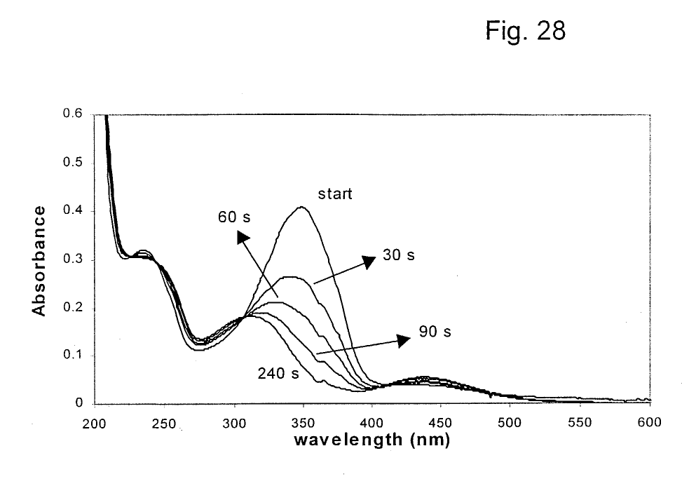

From the experiments it can be concluded that the isomerization cycle can be repeated several times without decomposition of the material. The trans azobenzene was subjected to irradiation (at 365 nm) for 30 second intervals, and the UV/Vis spectrum of the sample was taken between each irradiation cycle (FIG. 28). After 4 minutes of irradiation, the UV/Vis spectrum did not change, which points to a maximal isomerization to the cis isomer. As can be seen from FIG. 28, isobestic points are observed indicating that there is a transition from the trans isomer to the cis isomer and that there are no side reactions.

DSC experiments.

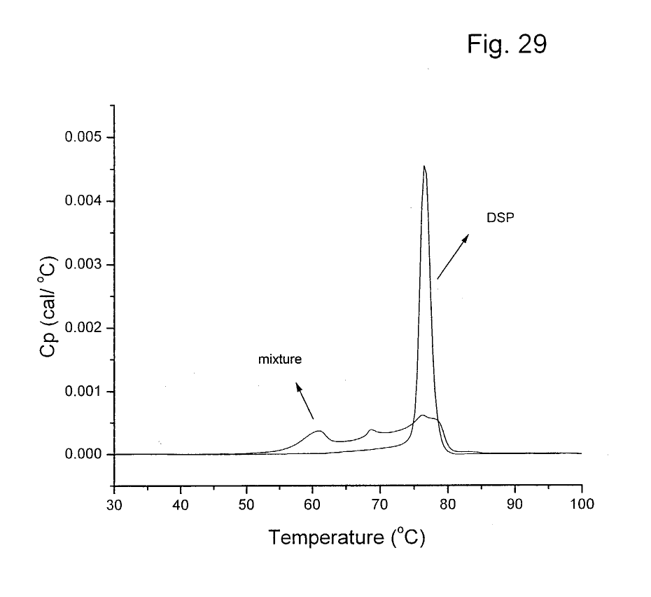

The DSC graphs show that the phase transition temperature of the vesicles of DSP is changed if 5% of lipid (mol. 6) was added (FIG. 29). This indicated that the azobenzene-containing lipids are incorporated into the vesicles. The broad transition indicates that a variety of domains of different lipid compositions are present.

The photoreactive lipids described above in combination with other lipids form liposomes and the physical properties of these liposomes can be altered upon irradiation. The MscL channel, or derivatives thereof, can be reconstituted into these lipid membranes and become responsive to the cis trans switching of the photoreactive lipids, resulting in controlled drug release.

### Example 6A

The design of an animal model and the testing of MscL-mediated drug release.

Besides triggered drug release (by pH, osmotic pressure and light), MscL-containing liposomes can be used for sustained drug release. The rate of drug release can be controlled by the rate of channel gating, a property that can be manipulated by genetic or chemical modification.

The effect of drug formulation on the rate of drug release in vivo was tested in the rat by external counting of radioactivity. The model was based on liposomal formulations that remain at the subcutaneous site of administration with an encapsulated model drug which, when released, was rapidly removed from the subcutaneous site of injection and excreted into the urinary bladder.

Materials and Methods

DOPC/DOPS (90:10, mol/mol) liposomes with or without the MscL mutant G22C were used (protein to lipid ratio of 1:20, wt/wt). Radiolabeled mertiatide (^{99m}Technetium-MAG3) was used as the model drug because of its rapid and exclusive excretion from the circulation into the urine (via active tubular secretion, 600 mL/min. in humans).

Sample preparation

Encapsulation of MAG3 in liposomes was performed by freezing/thawing three times and extrusion through a 400 nm filter. The free fraction of the compound was removed by G60 Sephadex column separation.

The normal liposomes were loaded with MAG3 in 0.9% NaCl and eluted on the G50 column with 25 mM HEPES pH 8 and 150 mM NaCl. The G22C-MscL-liposomes were loaded with the model drug in 150 mM sucrose and 145 mM NaCl and eluted on the G50 column with 25 mM HEPES pH 8, 150 mM sucrose, and 145 mM NaCl.

Kinetics of encapsulated model drugs administered subcutaneously.

Male Wistar rats were anaesthetized using iso-flurane O2/NO throughout the study. Liposome encapsulated MAG3 in a 0.5 ml volume was injected subcutaneously (in the neck) and accumulation of radioactivity in the urinary bladder was constantly monitored by external counting using a gamma-camera (window: 140 keV, 250 keV width, 1 min. time resolution). At the end of the study (after 40 min.), a 10 times higher dose of free MAG3 was administered subcutaneously or intravenously to measure the urinary excretion rates of those formulations.

Results and Discussion

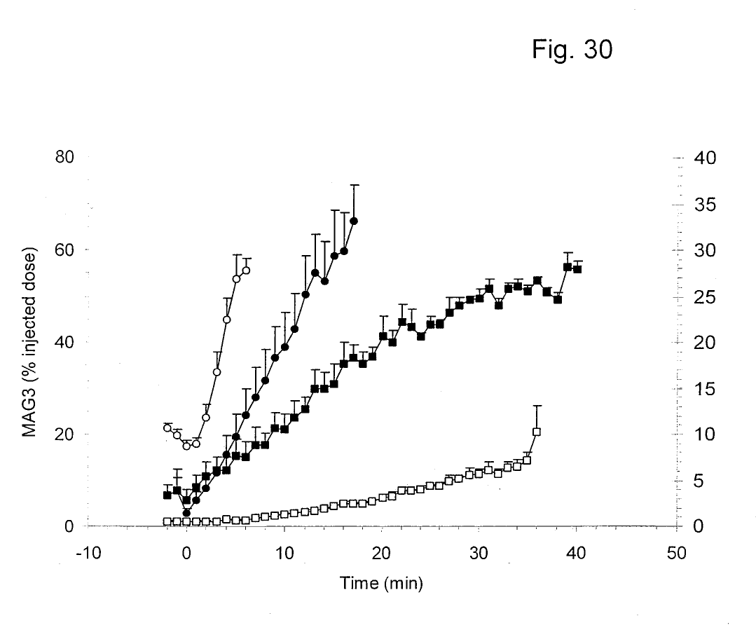

Kinetics of free and encapsulated model drugs (FIG. 30).

Compared to intravenous administration of the free compound, the urinary excretion of MAG3 was slower after subcutaneous injection (50% in 3 min. and >12 min., respectively).

Encapsulation in liposomes reduced the rate of urinary MAG3 excretion with a significant difference between the liposomes tested. Compared to the normal DOPC/DOPS liposomes, the G22C-MscL-containing liposomes released significantly more MAG3 (15% and 45% urinary MAG3 excretion in the first 30 min. after injection).

These results show that liposomes containing MscL mutant G22C exhibit release of the hydrophilic molecules MAG3. This MscL mediated release is significantly faster than MAG3 release from liposomes without MscL but slower compared to free MAG3 injected subcutaneously. Therefore, it can be concluded that the MscL channel modulates the transport of hydrophilic molecules in vivo, and can be used as a drug delivery vehicle for sustained release.

### Example 6B

Example 6B describes the use of MscL channels or derivatives thereof for sustained release of drugs. In examples 1, 2, 4, and 5, different examples are described to control the gating of the channel and thus the release of drugs. In one example, pH is described as a signal to control the gating of the channel. This example describes a method to induce a temporary pH-reduction subcutaneously for the testing of pH-sensitive MscL-mediated drug release.

Materials and Methods

pH-reduction subcutaneously.

Male Wistar rats remained conscious throughout the study. The subcutaneous pH was constantly recorded using a microglass electrode. After stabilization of the pH, 0.5 ml MES buffer (pH 6.1) was injected approximately 3 mm from the pH electrode. The effect of different molarities (50, 100 and 250 mM) of the MES buffer on the time-course of pH reduction was tested.

Results and Discussion

pH reduction subcutaneously.

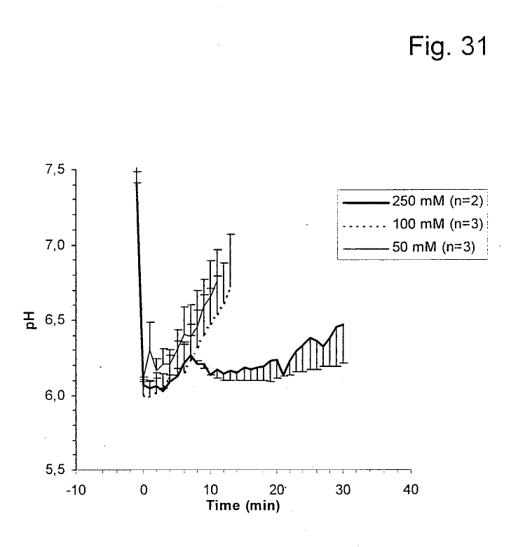

MES buffer is suitable to lower the pH in the subcutaneous tissue. The duration of pH reduction appeared to depend on the molarity of the buffer (FIG. 31). With 50 and 100 mM MES, the pH returned steadily to physiological pH (pH 7.4) within 10 min. By using 250 mM MES, the pH remained below pH 6.5 for more than 30 min.

The subcutaneous tissue can temporarily be acidified by an MES buffer with the molarity of the buffer determining the duration of pH reduction. Short-lasting pH reductions allow the measurement of the effect of repeated gating and closing of the channel.

### Example 6C

The radioactive method, described in Example 6A, is suitable to determine the rate of a subcutaneously released drug administered in different formulations. Drawbacks are the unphysiological state of anesthesia and the limited period of time that can be measured (due to the short half-life of the radioactive label, the instability of the compound and the required anesthesia). Therefore, an alternative was developed. In this method, the subcutaneous release of a drug from different formulations, can be determined in conscious rats for a long period of time (days).

Materials and Methods

DOPC/DOPS (90:10, mol/mol) and DOPC/DOPE liposomes (70:30, mol/mol) were tested. Iodo-thalamate (IOT) was chosen as a model drug because of its rapid and exclusive excretion via glomerular filtration, at 1 ml.min⁻¹.100 g rat, from the circulation into the urine.

Sample preparation.

Encapsulation of IOT in liposomes was performed by freezing/thawing three times followed by extrusion through a 400 nm filter. The free fraction of the compound was removed by G50 Sephadex column separation.

The liposomes were loaded with IOT in an iso-osmotic solution (25 mM HEPES ph 7.4 and 145 mM NaCl) and eluted on the G50 column using the same buffer as the eluens.

Kinetics of encapsulated model drugs administered subcutaneously.

Male Wistar rats remained conscious throughout the study. To increase the resolution time, the diuretic furosemide (10 mg/kg dose s.c.) was given in the morning, 7 hours before administration of the sample. Urine was collected automatically with a 1 hour resolution. The concentration of IOT in the urine was measured by HPLC.

Results and Discussion

Kinetics of free and encapsulated model drugs.

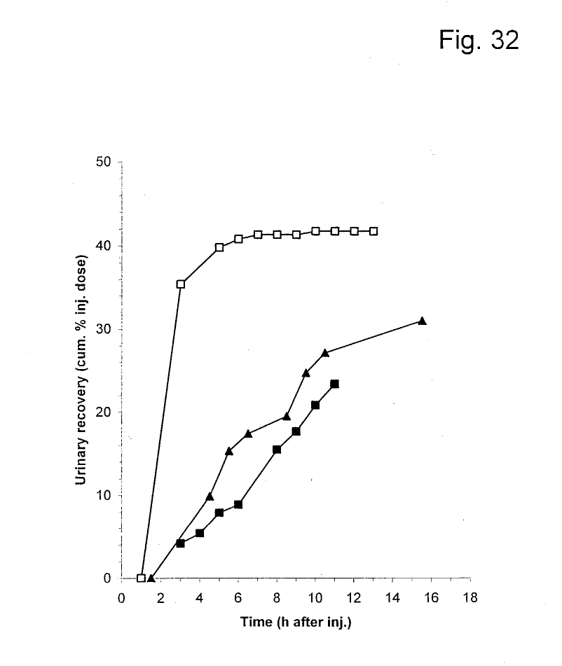

After subcutaneous injection of free IOT, the first phase urinary excretion of the model drug was completed in the first couple of hours after injection (FIG. 32). In contrast, the rate of excretion was strongly reduced by liposomal encapsulation.

Both the radioactive method (Example 6A) and the present method are suitable to measure the stability of subcutaneous liposomal drug formulations. The radioactive method is more suitable for relatively fast releasing formulations whereas the last described method is more suitable for the slower releasing formulations. These animal models can be used to monitor the controlled release of drugs from liposomal formulations containing MscL channels or derivatives thereof that respond to changes in pH, light of specific wavelengths, changes in osmolality, or the addition of an activator such as MTSET or reduced glutathione (described in previous examples).

### Example 7

MscL^{Ll}: Mechanosensitive channels of large conductance homologue found in Lactococcus lactis IL1403 (NCBI: 12725155).

For ease of explanation, superscript ^{Ll} will be used to refer to Lactococcus lactis and the superscript ^{Ec} will be used to refer to E. coli. Certain applications require MscL channels with specific characteristics. For these specific applications it is possible to use mutants or chemically modified MscL channels of E. coli. Alternatively, homologous mechanosensitive channels from other organisms could be used. This example describes the cloning, overexpression, purification, membrane reconstitution and functional characterization of an MscL homologue found in Lactococcus lactis. A result of this system is the significantly higher overexpression of the channel protein when the MscL channel protein originates, and is overexpressed, in a GRAS organism.

Material and Methods

MscL^{Ll} Expression, Purification and Reconstitution.

The gene of MscL^{Ll} was taken from the GRAS organism L. lactis IL1403 and cloned with a 6-histidine tag into an overexpression vector. L. lactis NZ9000 cells containing the plasmid pNZ8020MscL^{Ll}6H carrying the MscL-6Histidine construct were grown to OD₆₀₀ of approximately 1 in 3L M17 (Difco) medium supplemented with 10 mM arginine and 0.5% galactose and induced with 0.5 ng/ml (final concentration) Nisin for 3 h. The cells were harvested and washed by centrifugation (10 min. 6,000×g) in 50 mM Tris-HCl pH 7.3 buffer. After incubation for 30 min. at 30° C. with 10 mg/ml lysozyme, MgSO₄ was added to the cell suspension to a final concentration of 10 mM. DNase and Rnase were added to a concentration of 0.1 mg/ml and the cells were ruptured by two-fold passage through a French Pressure cell (15 k Psi L. lactis). The cell-debris and cell membranes were separated by centrifugation (10 min. at 11,000×g) after addition of 15 mM Na-EDTA at pH 7.0. The membranes, contained in the supernatant, were collected by ultra-centrifugation (1 h. at 150,000×g) and resuspended in 3 ml (total protein content: 20 mg/ml) 50 mM Tris-HCl, pH 7.3, and stored at −80° C. until further use.

Before purification, 1 volume of membranes was solubilized with 9 volumes of 50 mM Na₂HPO₄.NaHPO₄, 300 mM NaCl, 10 mM imidazole at pH 8.0 and buffer A containing 3% n-octyl β-glucoside. The extract was cleared by ultra-centrifugation (20 min. at 150,000×g) and mixed with 1 bed volume Ni²⁺-NTA agarose beads (pre-equilibrated with buffer A+1% n-octyl β-glucoside) and gently rotated for 30 min. at 4° C. The mixed column material was poured into a Bio-spin column (Bio-Rad) and washed with 20 volumes buffer A containing 1% n-octyl β-glucoside. The protein was eluted with buffer A containing 1% n-octyl β-glucoside and increasing amounts of L-Histidine (1 vol. 50 mM, 1 vol. 100 mM, 2×1 vol. 200 mM). Protein concentration was determined according to Schaffner and Weissmann (Shaffner et al.). Further analysis was done on an SDS-15% polyacrylamide gel followed by staining with Coomassie Brilliant Blue or transferral to PVDF membranes by semi-dry electrophoretic blotting for immunodetection with anti-His antibodies (Amersham Pharmacia Biotech). Immunodetection was performed with an alkaline phosphatase conjugated secondary antibody as recommended by the manufacturer (Sigma).

The purified protein was reconstituted with a mixture of the following lipids: 1,2-Dioleoyl-sn-Glycero-3-Phosphocholine (Avanti 850375) and 1,2-Dioleoyl-sn-Glycero-3-Phospho-L-serine (Avanti 810225) 9:1 w/w or Dioleoyl-sn-Glycero-3-Phosphocholine and Cholesterol (Avanti 700000) 8:2 mol:mol. Before reconstitution, the lipids were washed and mixed in chloroform (20 mg/ml) and dried under N₂ gas. The dried lipids were resuspended in 50 mM Kpi buffer at pH 7.0 to a final concentration of 20 mg/ml. The suspension was sonicated using a tip sonicator (8 cycles, 15s on 45s off, intensity of 4 μm (peak to peak)). The formed liposome solution was completely solubilized using n-octyl β-glucoside and the purified protein was added (1:1000, 1:500 or 1:50 w/w protein/lipid). Proteoliposomes were formed by dialyzing the lipid-protein mixture for 3 days at 4° C. against 500 volumes 50 mM Kpi at pH 7.0 without any detergent, using a 3,500 Da MWCO Spectrum spectrapor dialysis membrane. After the first night of incubation, 0.5 g of polystyrene beads (Bio-Beads SM2™) were added for extra detergent removal.

Freeze Fracture Electron Microscopy.

Freeze fracture electron microscopy of reconstituted MscL^{Ll} was performed as described elsewhere (Friesen et al.).

Electrophysiological characterization of MscL^{Ll}.

Electrophysiological characterization was essentially performed as described by Blount et al. (Blount et al.). Giant spheroplasts of E. coli PB104 (MscL negative) containing the plasmid pB10bMscL^{Ll}6H (for overexpression of MscL^{Ll}) were generated. Cells were grown to OD₆₀₀ of 0.5 diluted 10 fold and grown in the presence of 60 μg/ml cephalexin (preventing septation, but not cell growth) and 1.3 mM IPTG. When the cells had formed non-septated filamentous snakes of 50-150 μm, they were harvested at 5,000×g. The pellet was resuspended in 1/10^{th} of the original volume of 0.8M sucrose. Cell outer membranes (peptidoglycan) were digested with lysozyme (200 μg/ml) in the presence of DNase (50 μg/ml) in 50 mM Tris-HCl, 6 mM Na-EDTA at pH 7.2 for 2-5 minutes. The reaction was stopped when sufficient giant spheroplasts were formed by the addition of 8 mM MgCl₂ (final concentration). Spheroplasts were enriched by spinning on a 0.8 M sucrose cushion.

Alternatively, patches were studied from liposomes with reconstituted MscL^{Ll}. Proteoliposomes were centrifuged at 200,000×g, resuspended to 100 mg/ml in 10 mM MOPS pH 7.2, 5% ethylene glycol and dried overnight for 4 h on glass slides in a desiccator at 4° C. Rehydration of the lipids to 100 mg/ml was performed in liposome patch buffer (200 mM KCl, 0.1 M EDTA, 10⁻² mM CaCl₂ and 5 mM HEPES, pH 7.2). The proteoliposomes were loaded into the sample well containing patch buffer with 20 mM MgCl₂, causing the lipid sample to form large unilamellar blisters, which were patched.

Patches were examined at room temperature, with symmetrical solutions for pipette and bath. The buffer includes 200 mM KCl, 0.1 M EDTA, 10⁻² mM CaCl₂ and 5 mM HEPES, pH 7.2. For spheroplasts 90 mM of MgCl₂ was added, and for proteoliposomes 20 mM of MgCl₂ was added. Recordings of current through open channels were performed at +/−20 mV. Data on pressure and current were acquired at a sampling rate of 30 kHz with a 10 kHz filtration and analyzed using PCLAMP8 software. Pressure was measured using a piezoelectric pressure transducer (Micro-switch) (World Precision Instruments PM01).

In vitro release profiles of a model drug from proteoliposomes as described in example 1.

Results and Discussion

MsCL^{Ll} Expression, Purification and Reconstitution.

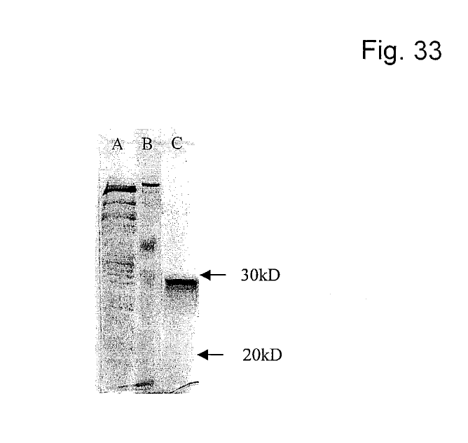

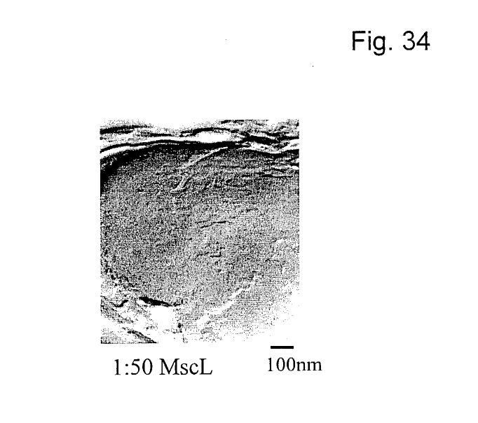

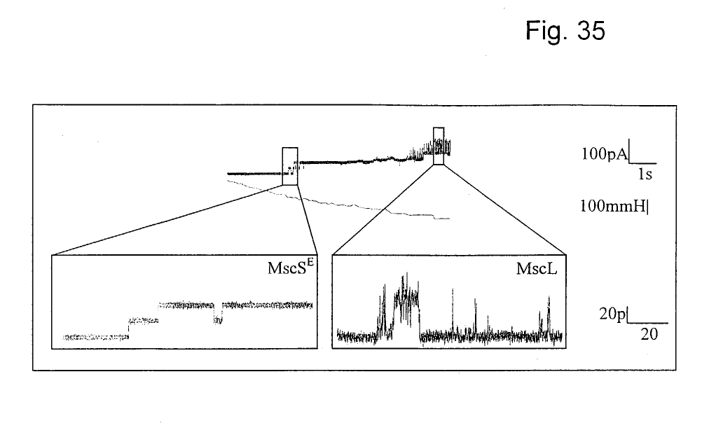

Expression levels in the membrane were around 5% of total membrane protein. MscL^{Ll} was purified to apparent homogeneity in a single step using nickel chelate affinity chromatography as shown in FIG. 33 lane C. Per ml of vesicles, about 1 mg of protein could be obtained at an estimated purity of 95% based on SDS-PAGE and Coomassie Brilliant Blue staining. The band also showed up very clearly after immunological detection on a PVDF membrane (data not shown). The amount of protein for reconstitution was determined experimentally. For patch-clamp experiments, a 1:1000 w/w protein/lipid ratio was found to be useful, whereas for the calcein release assay, a 1:500 ratio seemed to give the clearest results.

Freeze Fracture Electron Microscopy.

FIG. 34 shows an electron micrograph of freeze-fractured proteoliposomes. As can be seen, the MscL^{Ll} protein was indeed inserted into the lipid bilayer.

Electrophysiological characterization of MscL^{Ll}.

FIG. 35 shows a typical trace of the MscL^{Ll} in E. coli spheroplasts. The channel openings are indicated as an upward current as a result of the applied pressure. Both MSCS^{Ec} and MscL^{Ll} channels are visible in this patch, enabling a sensitivity comparison to MsCL^{Ec}. This showed that MscL^{Ll} in E. coli cells opens at higher pressures than MscL^{Ec}. The ratio of pressures for opening MscL/MscS is 2.4 for MscL° C. and 2.8 for MscL^{Ll}.

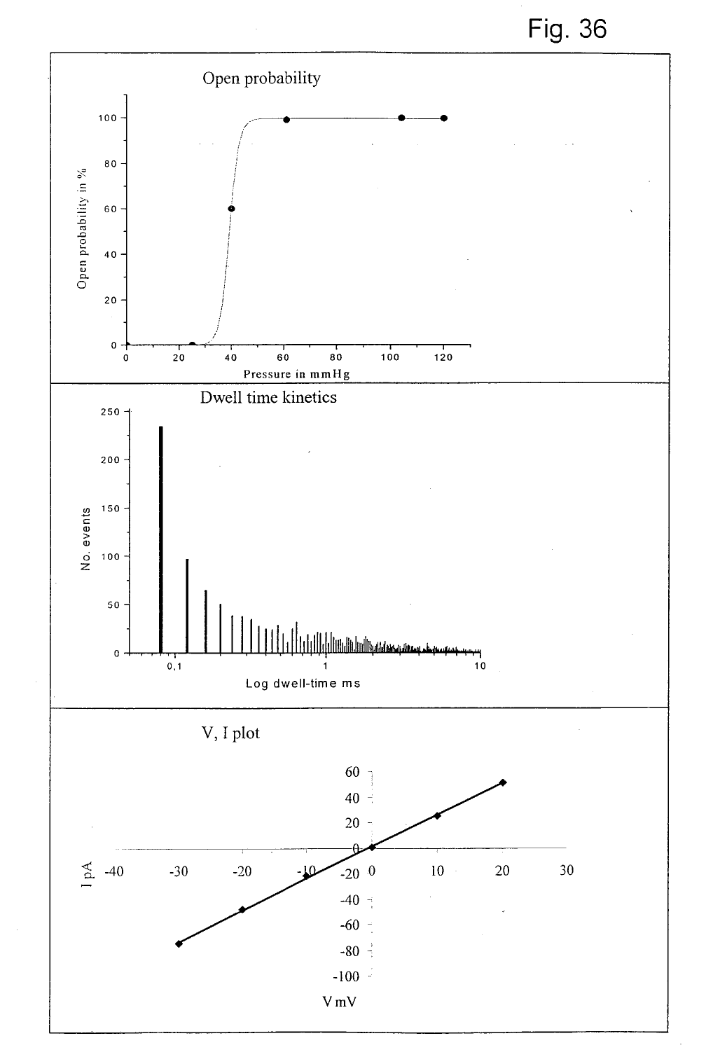

FIG. 36 provides information on pressure sensitivity, open dwell time and conductance of MscL^{Ll} which are all comparable to the values found for MsCL^{Ec}.

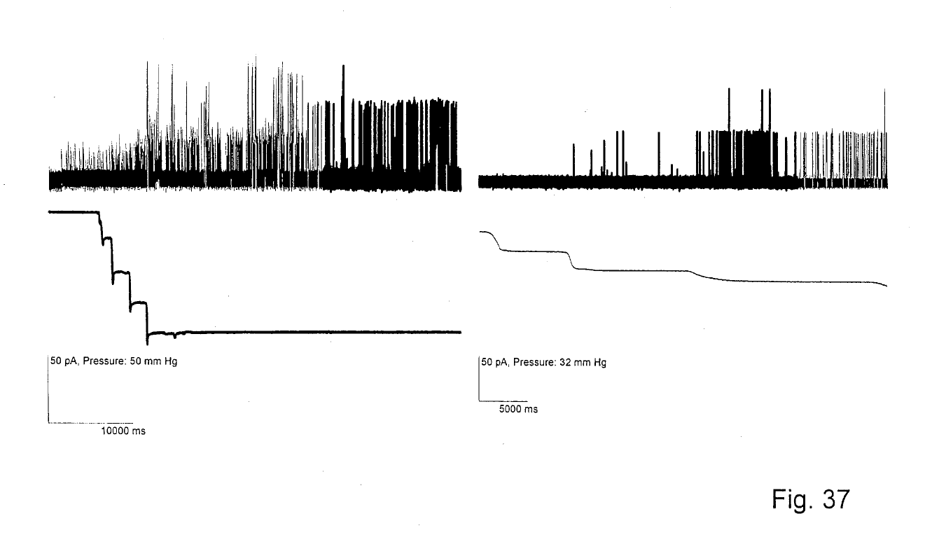

FIG. 37 shows traces of MscL^{Ll} reconstituted into different lipid compositions. The initial full openings occur at different pressures in the different liposome compositions.

In vitro release profiles of a model drug from proteoliposomes.

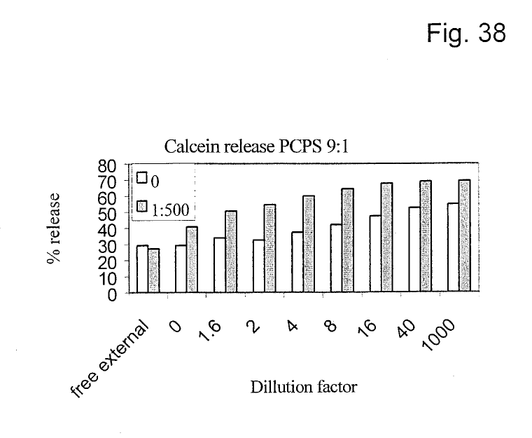

FIG. 38 shows the release of calcein in response to an osmotic shock in proteoliposomes containing MscL^{Ll}. The results of patch clamp and the calcein release assay show that this MscL homologue can be used to deliver substances from liposomes as described for MscL from E. coli and derivatives thereof.

### Example 8

Controlled release of Insulin from liposomes containing MscL Mutant G22C.

The present invention provides a method for obtaining controlled release of hydrophilic drugs from liposomes. For practical reasons, either calcein release or ion fluxes are monitored to functionally characterize the delivery system. This example shows that the observed principles in the previous examples also apply to therapeutically relevant hydrophilic molecules. Additionally, the applied filter-binding assay can be used to test the controlled release of many different substances from these delivery vehicles.

Materials and Methods

DOPC:DOPS (9:1 mol/mol) liposomes containing the G22C MscL were prepared as described in example 2. Insulin and fluorescein isothiocyanate (FITC) were obtained from Sigma (St. Louis, Mo., USA). Insulin (23 mg) was reacted with a four-fold molar ratio of FITC in 0.1 N borate buffer, pH 9.0, for 60 min. The pH was lowered to 7.5 with 0.1 N boric acid and the solution was extensively dialyzed, using a dialysis membrane with a molecular weight cut-off of 2,000 Da, for 96 hours against water at 4° C. with frequent water changes. Absorption spectra of the dialyzed sample were used to quantify the protein concentration and the stoichiometry of labeling. Concentrations of FITC and insulin were both 0.1 mM. The labeled insulin was encapsulated by three freeze-thaw cycles, followed by extrusion through a 400 nm polycarbonate membrane. The proteoliposomes containing labeled insulin were separated from free labeled insulin by using sephadex G-50 column chromatography equilibrated with 145 mM NaCl, 300 mM Sucrose, 25 mM Tris.HCl and 1 mM EDTA, pH 8.0.

Proteoliposomes were prepared as described in example 2 and MTSET was used for opening of the MscL mutant G22C channels. Samples were taken at different time points and Triton was added as a control for maximum fluorescence (100%). Samples were filtered over a 450 nm Cellulose Nitrate filter (Schleicher & Schuell BA85). The filtrate of 2 ml was retained and the fluorescence of 200 I11 of each filtrate was monitored in an fl600 plate reader (Bio-Tek). All experiments were performed in triplicate.

Results and Discussion

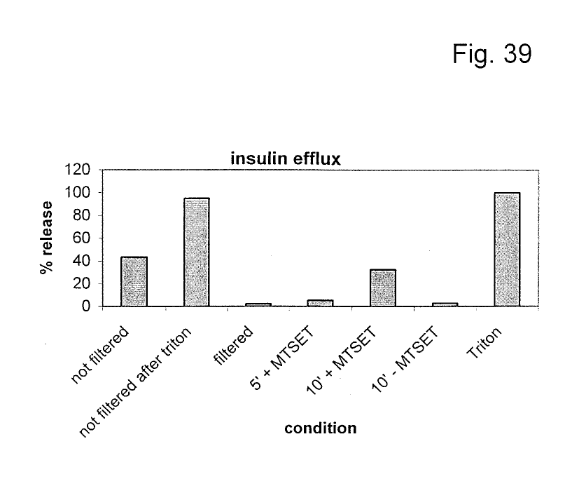

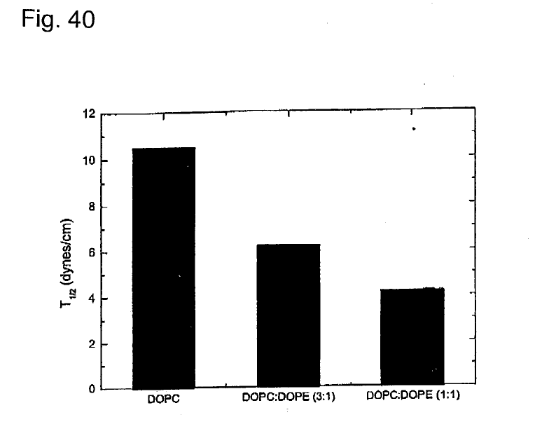

FIG. 39 shows the release of FITC-insulin through MscL mutant G22C upon activation with 1 mM MTSET. The difference between filtered and unfiltered conditions is the amount of FITC-insulin encapsulated in the proteoliposomes. The fluorescence of the unfiltered condition with and without Triton X-100 indicates that the concentration of FITC-insulin in the proteoliposomes exhibits self-quenching. Control conditions with MTSET and without MTSET were used to determine the effect of MTSET on the FITC-insulin efflux and to show that FITC-insulin efflux is indeed MscL mediated.

The mass of FITC-insulin is approximately 6,100 Da and considerably higher compared to calcein. Therefore, this example shows the applicability of this delivery system for therapeutic macromolecules. This filter assay can also be used for monitoring the controlled release of other labeled drug molecules from proteoliposomes.

### Example 9

Induced opening of the MscL channel by specific recognition.

Three peptides, spanning the portion of the channel accessible at the exterior of the liposomes, were synthesized and used to raise antibodies in rabbits. The bleeds from these rabbits contained antibodies specific for the synthesized peptides and the full length MscL. Single channel electrophysiologic characterization showed that the bleeds contained antibodies that specifically recognize MscL in the open formation. The antibodies were used to shift the conformational equilibrium to the open state of the channel.

### REFERENCES

Arkin, I. T., Sukharev, S. I., Blount, P., Kung, C., and Briinger A. T. (1998) Biochim. Biophys. Acta 1369, 131-140.

Delcour, A. H., Martinac, B., Adler, J., and Kung, C. (1989) Biophys. J. 56, 631-636.

Blount, P., Sukharev, S. I., Moe, P. C., Schroeder, M. J., Guy, H. R., and Kung, C. (1996) EMBO J. 15, 4798-4805.

Van Montfort, B. A., Cana, B., Duurkens, R., Godovac-Zimmermann, J., and Robillard, G. T., submitted.

Hirel, A. U., Schmitter, M. J., Dessen, P., Fayat, G., and Blanquet, S. (1989) Proc. Natl. Acad. Sci. U.S.A. 86, 8247-8251.

Knol, J., Sjollema, and Poolman, B. (1998) Biochemistry 37, 16410-16415.

Friesen, R. H. E., Knol, J., Poolman, B. (2000) J. Biol. Chem. 43, 33527-33535.

Moe, P. C., Blount, P. and Kung, C. (1998) Mol. Microbiol. 28, 583-592.

Yoshimura, K., Batiza, A., Schroeder, M., Blount P., and Kung, C. (1999) Biophys. J 77, 1960-1972.

Song, X., Perlstein, J., and Whitten, D. G. (1997) J. Am. Chem. Soc. 119, 9144-9159.

Blount, P., Sukharev, S. I., Schroeder, M. J., Nagle, S. K., and Kung, C. (1996) Proc. Natl. Acad. Sci USA 93, 11652-11657.

Mayer, L. D., Hope, M. J., and Cullis, P. R. (1986) Biochim. Biophys. Acta 858, 161-168.

Martinac, B., Adler, J., and Kung, C. (1990) Nature 348, 261-263.

Ou, X., Blount, P., Hoffman, R. J., and Kung C. (1998) Proc. Natl. Acad. Sci. USA 95, 11471-11475.

Damen J., Regts, J., and Scherphof G. (1981) Biochim. Bioph. Acta 665, 538-545.

Yoshimura, K, Batiza and Kung, C. (2001) Biophysical Journal (80) 2198-2206.

Sakuragi, M.; Aoki, K.; Tamaki, T.; Ichimura, K. Bull. Chem. Soc. Jpn., 1990, 63, 74.

Bixler, R. L.; Niemann, C. J. Org. Chem., 1958, 23, 575.

Yankeelov, J. A.; Jolley, C. J. Biochemistry, 1972, 11, 159.

Maat, L.; Beyerman, H. C.; Noordam, A. Tetrahedron, 1979, 35, 273.

Lucas, L. N.; Esch, J.; Kellog, R. M.; Feringa, B. L. Chem. Commun., 1998, 2313.

W. Schaffner and C. Weissmann. A rapid, sensitive, and specific method for the determination of protein in dilute solution. Anal. Biochem. 56 (2):502-514, 1973.

R. H. Friesen, J. Knol, and B. Poolman. Quaternary structure of the lactose transport protein of Streptococcus thermophilus in the detergent-solubilized and membrane-reconstituted state. J. Biol. Chem. 275 (43):33527-33535, 2000.

P. Blount, S. I. Sukharev, P. C. Moe, B. Martinac, and C. Kung. Mechanosensitive channels of bacteria. Methods Enzymol. 294:458-482, 1999.

What is claimed is:

1. A method for delivering a small hydrophilic molecule to a cell, said method comprising:
providing a lipid vesicle comprising a proteinaceous channel, wherein said proteinaceous channel, in an open state, allows passage of small hydrophilic molecules therethrough to said lipid vesicle's exterior, said lipid vesicle formulated such that, upon activation, said proteinaceous channel opens;
loading said lipid vesicle with a small hydrophilic molecule;
administering said lipid vesicle to a fluid in contact with the cell;
allowing said lipid vesicle to migrate to said cell's vicinity;
activating said lipid vesicle; and
thus opening said proteinaceous channel and delivering said small hydrophobic molecule to the cell.

2. The method according to claim 1, wherein administering said lipid vesicle comprises administering said lipid vesicle to a subject.

3. The method according to claim 1 or claim 2, wherein said proteinaceous channel is a solute channel.

4. The method according to any one of claims 1-3, wherein said proteinaceous channel is a mechanosensitive channel.

5. The method according to claim 4, wherein said proteinaceous channel is a mechanosensitive channel of large conductance (MscL).

6. The method according to any one of claims 1-5, wherein said lipid vesicle comprises positively or neutrally charged lipids.

7. The method according to claim 6, wherein said lipid vesicle consists essentially of positively or neutrally charged lipids.

8. The method according to any one of claims 1-7, wherein said proteinaceous channel is a mutant mechanosensitive channel of large conductance.

9. The method according to any one of claims 1-8, wherein the small hydrophilic molecule is a peptide.

10. The method according to any one of claims 1-9, wherein said small hydrophilic molecule has a diameter smaller than about 60 A.

11. The method according to claim 10, wherein said small hydrophilic molecule has a diameter smaller than 40 A.

12. The method according to any one of claims 1-11, wherein said proteinaceous channel is activated by a signal.

13. The method according to claim 12, wherein the signal is selected from the group consisting of a light signal, an altered pH, temperature change, or mixture of any thereof.

14. The method according to claim 13, wherein the signal is an altered pH of less than or equal to about 6.5.

15. A composition for delivering a small hydrophilic molecule to a target cell, said composition comprising:
a lipid vesicle comprising the small hydrophilic molecule and a proteinaceous channel having open and closed states; and
wherein said composition is formulated such that said proteinaceous channel is normally in the closed state thus retaining the small hydrophilic molecule therein.

16. The composition of claim 15, wherein said proteinaceous channel comprises a mechanosensitive channel of large conductance.

17. The composition of claim 15 or claim 16, wherein said lipid vesicle comprises an asymmetrical bilayer.

18. The composition of any one of claims 16-17, wherein said lipid vesicle comprises a light-sensitive lipid or a light-sensitive mechanosensitive channel of large conductance.

19. The composition of any one of claims 16-18, wherein said proteinaceous channel opens in response to a stimulus selected from the group consisting of light, local relative pH increase or decrease, local relative temperature increase or decrease, and a mixture of any thereof.

20. The composition of any one of claims 16-19, wherein the stimulus that opens said proteinaceous channel is provided by an intermediate.

21. The composition of any one of claims 16-19, wherein said lipid vesicle comprises a neutral lipid.

22. The composition of any one of claims 16-19, wherein said lipid vesicle comprises a positively charged lipid.

23. The composition of claim 21 or claim 22, wherein said lipid vesicle does not comprise a negatively charged lipid.

24. The composition of any one of claims 16-23, wherein the small hydrophilic molecule is capable of passing through an activated mechanosensitive channel of large conductance (MscL).

25. The composition of any one of claims 15-24, further comprising a non-channel protein.

26. The composition of claim 25, wherein said non-channel protein is a binding molecule capable of binding to a binding partner on the target cell, wherein said binding enables at least a prolonged stay of said lipid vesicle near the target cell.

27. A method of delivering a small hydrophilic molecule to a target tissue in a subject, said method comprising:
providing a lipid vesicle comprising a mechanosensitive channel of large conductance (MscL) and small hydrophilic molecule; and
parenterally administering said lipid vesicle to the target tissue.

28. A lipid vesicle for modulating the bio-availability of a small hydrophilic molecule upon administration of said lipid vesicle to a subject, said lipid vesicle comprising:
a small hydrophilic molecule;
a proteinaceous channel incorporated into said lipid vesicle wherein an open state of said proteinaceous channel allows passage of said small hydrophilic molecule therethrough to said lipid vesicle's exterior.

29. The lipid vesicle of claim 28, wherein said proteinaceous channel is a mechanosensitive channel.

30. The lipid vesicle of claim 29, wherein said mechanosensitive channel is a mechanosensitive channel of large conductance (MscL).
